(19) United States
(12) Patent Application Publication
(10) Pub. No.: US 2010/0273924 A1
(43) Pub. Date: Oct. 28, 2010

(54) CURABLE COMPOSITIONS HAVING LESS VOLATILIZATION

(75) Inventor: Urs Burckhardt, Zurich (CH)

Correspondence Address: OLIFF & BERRIDGE, PLC, P.O. BOX 320850, ALEXANDRIA, VA, 22320-4850, US

(73) Assignee: SIKA Technology AG, Baar (CH)

(21) Appl. No.: 12/808,968

(22) PCT Filed: Dec. 19, 2008
(86) PCT No.: PCT/EP08/67985

§ 371 Date: Jun. 17, 2010

(30) Foreign Application Priority Data
Dec. 21, 2007 (EP) 07150364.3

(51) Int. Cl. C08K 5/25; B05D 3/02; C08K 5/43; B32B 37/12; C07C 243/10

(52) U.S. Cl. 524/189; 427/385.5; 524/168; 156/331.1; 564/464

(57) ABSTRACT

The invention particularly relates to curable compositions comprising at least one polyisocyanate, at least one amine that is blocked by means of aldehyde or ketone, and at least one carboxylic acid hydrazide or sulfonic acid hydrazide which has a minimum melting point of 100° C., especially at least 150° C. Surprisingly, after being cured using moisture at room temperature and when being heated to increased temperatures, significantly fewer volatile components of such compositions volatilize than in the corresponding compositions that do not contain said hydrazide.

### TECHNICAL FIELD

The present invention pertains to the field of moisture-curing polyurethane compositions and also to their use, more particularly as elastic adhesives in vehicle construction.

### PRIOR ART

Curable compositions containing isocyanate groups, also referred to as polyurethane compositions, have been used for a long time in a wide variety of applications, including as one-pack and two-pack elastic adhesives, sealants or coatings. For the better curing of such compositions, they may be admixed with moisture-activable amine crosslinkers, referred to as “blocked amines” or “latent curing agents”, which largely prevent the direct, carbon dioxide (CO₂)-producing reaction of the isocyanate groups with moisture and hence the formation of unwanted gas bubbles (blisters) in the cured composition.

The use of blocked amines, however, may also cause problems not least in view of the fact that the curing reaction produces aldehydes or ketones. These elimination products as they are called, are not incorporated into the polyurethane matrix, and can therefore escape to the environment by evaporation or migration. The aldehydes or ketones produced from the blocked amines often represent substances which are intensely odorous and/or a burden to health.

Particularly in the case of interior applications, the outgassing of organic substances from, for example, an adhesive into the surrounding air can be a problem. Volatile substances accumulate rapidly in the inside air and can, particularly if they are intensely odorous, provoke nausea or respiratory tract irritation. Less volatile, low-odor substances are usually not noticeable immediately; however, they may deposit by condensation on surfaces and so gradually lead to the formation of mists, as for example on glazing. Within the automobile industry, the formation of such mists, which can lead to disruptive clouding, is referred to as “fogging”. In materials such as adhesives, for example, which are intended for use in a vehicle interior, there are generally strict yardsticks applied concerning substances which may outgas.

WO 2004/013088 A1 and WO 2007/036571 A1 disclose blocked amines in the form of odorless aldimines and polyurethane compositions comprising such aldimines. The aldehydes released when the compositions are cured are odorless compounds of relatively low volatility which at room temperature remain largely in the cured composition. At elevated temperature, however, there may nevertheless be outgassing of these aldehydes from the composition.

DE 1 156 552 and U.S. Pat. No. 6,051,620 disclose polyurethane compositions comprising hydrazide. In these systems the hydrazides used are reacted prior to or during the reaction of the isocyanate groups.

### SUMMARY OF THE INVENTION

It is an object of the present invention, therefore, to provide a polyurethane composition which has the advantages of the compositions known from the prior art which cure by means of blocked amines, but in the cured state exhibit significantly reduced outgassing of aldehydes or ketones, more particularly at elevated temperature, as for example at 80° C. or more.

Surprisingly it has been found that curable compositions according to claim 1 which as well as an aldehyde-blocked or ketone-blocked amine comprise a hydrazide of a carboxylic or sulfonic acid that has a sufficiently high melting point achieve this object. These compositions cure with moisture at room temperature or slightly elevated temperature, more particularly below 40° C., without significant participation of the hydrazide, producing aldehydes and/or ketones. When the cured compositions are heated, more particularly to 80° C. or more, the hydrazide begins to react with the resultant aldehydes and/or ketones, to form condensation products which are of low volatility and which do not outgas even at high temperatures. The cured compositions therefore have advantages in particular in the context of fogging behavior.

Further provided by the invention are a method of adhesive bonding according to claim 19, of sealing according to claim 18 and of coating according to claim 21; the resultant articles according to claim 22; and a cured composition according to claim 23. Finally, further aspects of the present invention are formed by an aldazide according to claim 24, and by the use of a hydrazide according to claims 28 and 29.

Preferred embodiments of the invention are the subject matter of the dependent claims.

### SOME EMBODIMENTS OF THE INVENTION

The invention provides a curable composition comprising
a) at least one polyisocyanate P,
b) at least one aldehyde or ketone-blocked amine BA,
c) at least one hydrazide HY of a carboxylic acid or sulfonic acid, which has a melting point of at least 100° C., more particularly of at least 150° C.,

with the proviso that the hydrazide HY is present in an amount of 0.3 to 1.1 equivalents of hydrazide groups per equivalent of aldehyde groups or keto groups, with which the amine BA is blocked.

The term “hydrazide” in the present document identifies the condensation product of a carboxylic or sulfonic acid and hydrazine.

The curable composition described comprises at least one polyisocyanate P.

The term “polyisocyanate” in the present document encompasses compounds having two or more isocyanate groups, independently of whether they are monomeric diisocyanates, oligomeric polyisocyanates, or polymers containing isocyanate groups and having a relatively high molecular weight.

The term “polymer” in the present document encompasses on the one hand a collective of macromolecules which, while being chemically uniform, nevertheless differ in respect of degree of polymerization, molar mass, and chain length, and have been prepared by a polymerization reaction (chain-growth addition polymerization, polyaddition, polycondensation). The term also, moreover, encompasses derivatives of such a collective of macromolecules from polymerization reactions, in other words compounds which have been obtained by means of reactions, such as additions or substitutions, for example, of functional groups on existing macromolecules, and which may be chemically uniform or chemically non-uniform. The term also encompasses, furthermore, what are known as prepolymers, by which are meant reactive oligomeric pre-adducts whose functional groups take part in the construction of macromolecules.

In one embodiment a suitable polyisocyanate P is a polyurethane polymer PUP containing isocyanate groups.

The term “polyurethane polymer” encompasses all polymers which are prepared by the process known as the diisocyanate polyaddition process. This also includes those polymers which are entirely or virtually free from urethane groups. Examples of polyurethane polymers are polyether-polyurethanes, polyester-polyurethanes, polyether-polyureas, polyureas, polyester-polyureas, polyisocyanurates and polycarbodiimides.

One suitable polyurethane polymer PUP is obtainable more particularly from the reaction of at least one polyol with at least one polyisocyanate. This reaction may involve the polyol and the polyisocyanate being reacted by customary methods, at temperatures, for example of 50° C. to 100° C., optionally with accompanying use of suitable catalysts, the amount of the polyisocyanate being such that its isocyanate groups are present in a stoichiometric excess in relation to the hydroxyl groups of the polyol. The amount of the polyisocyanate is advantageously such that an NCO/OH ratio of 1.3 to 5, more particularly of 1.5 to 3, is observed. The “NCO/OH ratio” means the ratio of the number of isocyanate groups used to the number of hydroxyl groups used. After the reaction of all of the hydroxyl groups of the polyol, the polyurethane polymer PUP preferably retains a free isocyanate group content of 0.5 to 15% by weight, more preferably of 0.5 to 10% by weight.

The polyurethane polymer PUP may optionally be prepared with accompanying use of plasticizers, in which case the plasticizers used contain no isocyanate-reactive groups.

Polyols which can be used for preparing a polyurethane polymer PUP include, for example, the following commercially customary polyols or mixtures thereof:
polyoxyalkylene polyols, also called polyether polyols or oligoetherols, which are polymerization products of ethylene oxide, 1,2-propylene oxide, 1,2- or 2,3-butylene oxide, oxetane, tetrahydrofuran or mixtures thereof, optionally polymerized by means of a starter molecule having two or more active hydrogen atoms, such as, for example, water, ammonia or compounds having two or more OH or NH groups, such as, for example, 1,2-ethanedial, 1,2- and 1,3-propanediol, neopentylglycol, diethylene glycol, triethylene glycol, the isomeric dipropylene glycols and tripropylene glycols, the isomeric butanediols, pentanediols, hexanediols, heptanediols, octanediols, nonanediols, decanediols, undecanediols, 1,3- and 1,4-cyclohexane-dimethanol, bisphenol A, hydrogenated bisphenol A, 1,1,1-trimethylolethane, 1,1,1-trimethylolpropane, glycerol, aniline, and also mixtures of the aforementioned compounds. Use may be made not only of polyoxyalkylene polyols which have a low degree of unsaturation (measured in accordance with ASTM D-2849-69 and expressed in milliequivalents of unsaturation per gram of polyol (meq/g)), prepared, for example, by means of what are called double metal cyanide complex catalysts (DMC catalysts), but also of polyoxyalkylene polyols having a higher degree of unsaturation, prepared, for example, by means of anionic catalysts such as NaOH, KOH, CsOH or alkali metal alkoxides.

Particularly suitable are polyoxyalkylene diols or polyoxyalkylene triols, more particularly polyoxyethylene and polyoxypropylene dials and triols. Especially suitable are polyoxyalkylene dials and triols having a degree of unsaturation of less than 0.02 meq/g and having a molecular weight in the range of 1000-30 000 g/mol, and also polyoxypropylene dials and trials having a molecular weight of 400-8000 g/mol.

Likewise particularly suitable are what are called ethylene oxide-terminated (“EO-end capped”, ethylene oxide-end capped) polyoxypropylene polyols. The latter are special polyoxypropylene-polyoxyethylene polyols which are obtained, for example, by further alkoxylating pure polyoxypropylene polyols, more particularly polyoxypropylene dials and trials, with ethylene oxide after the end of the polypropoxylation reaction, and as a result contain primary hydroxyl groups.
Styrene-acrylonitrile or acrylonitrile-methyl methacrylate-grafted polyether polyols.
Polyester polyols, also called oligoesterols, prepared for example from dihydric to trihydric alcohols such as, for example, 1,2-ethanediol, diethylene glycol, 1,2-propanediol, dipropylene glycol, 1,4-butanediol, 1,5-pentanediol, 1,6-hexanediol, neopentylglycol, glycerol, 1,1,1-trimethylolpropane or mixtures of the aforementioned alcohols with organic dicarboxylic acids or their anhydrides or esters such as, for example, succinic acid, glutaric acid, adipic acid, suberic acid, sebacic acid, dodecanedicarboxylic acid, maleic acid, fumaric acid, phthalic acid, isophthalic acid, terephthalic acid and hexahydrophthalic acid or mixtures of the aforementioned acids, and also polyester polyols from lactones such as ε-caprolactone, for example.
Polycarbonate polyols, of the kind obtainable by reaction, for example, of the abovementioned alcohols—those used to construct the polyester polyols—with dialkyl carbonates, diaryl carbonates or phosgene.
Block copolymers which carry at least two hydroxyl groups and which contain at least two different blocks with polyether, polyester and/or polycarbonate structure of the type described above.
Polyacrylate polyols and polymethacrylate polyols.
Polyhydrocarbon polyols, also called oligohydrocarbonols, such as, for example, polyhydroxy-functional ethylene-propylene, ethylene-butylene or ethylene-propylene-diene copolymers, of the kind produced, for example, by the company Kraton Polymers, or polyhydroxy-functional copolymers of dienes such as 1,3-butanediene or diene mixtures and vinyl monomers such as styrene, acrylonitrile or isobutylene, or polyhydroxy-functional polybutadiene polyols, such as, for example, those which are prepared by copolymerization of 1,3-butadiene and allyl alcohol and which may also have been hydrogenated.
Polyhydroxy-functional acrylonitrile/butadiene copolymers, of the kind preparable, for example, from epoxides or amino alcohols and carboxyl-terminated acrylonitrile/butadiene copolymers (available commercially under the name Hycar® CTBN from Hanse Chemie).

These stated polyols preferably have an average molecular weight of 250-30 000 g/mol, more particularly of 400-20 000 g/mol, and preferably have an average OH functionality in the range from 1.6 to 3.

Preferred polyols are polyether, polyester, polycarbonate and polyacrylate polyols, preferably dials and triols. Particularly preferred are polyether polyols, more particularly polyoxypropylene polyols and polyoxypropylene-polyoxyethylene polyols.

Further to these stated polyols it is possible as well to use small amounts of low molecular mass dihydric or higher polyhydric alcohols, such as, for example, 1,2-ethanediol, 1,2- and 1,3-propanediol, neopentylglycol, diethylene glycol, triethylene glycol, the isomeric dipropylene glycols and tripropylene glycols, the isomeric butanediols, pentanediols, hexanediols, heptanediols, octanediols, nonanediols, decanediols, undecanediols, 1,3- and 1,4-cyclohexanedimethanol, hydrogenated bisphenol A, dimeric fatty alcohols, 1,1,1-trimethylolethane, 1,1,1-trimethylolpropane, glycerol, pentaerythritol, sugar alcohols such as xylitol, sorbitol or mannitol, sugars such as sucrose, other alcohols of relatively high hydricity, low molecular mass alkoxylation products of the aforementioned dihydric and polyhydric alcohols, and also mixtures of the aforementioned alcohols, when preparing the polyurethane polymer PUP. It is likewise possible for small amounts of polyols having an average OH functionality of more than 3 to be used, examples being sugar polyols.

Polyisocyanates used for preparing a polyurethane polymer PUP containing isocyanate groups are aromatic or aliphatic polyisocyanates, more particularly the diisocyanates.

“Aromatic isocyanate” identifies an organic compound which contains exclusively aromatic isocyanate groups. “Aromatic” identifies an isocyanate group which is attached to an aromatic or heteroaromatic radical. “Aliphatic isocyanate” identifies an organic compound which contains aliphatic isocyanate groups. “Aliphatic” identifies an isocyanate group which is attached to an aliphatic, cycloaliphatic or arylaliphatic radical.

Examples of suitable aromatic polyisocyanates include monomeric diisocyanates or triisocyanates such as 2,4- and 2,6-tolylene diisocyanate and any desired mixtures of these isomers (TDI), 2,4′- and 2,2′-diphenylmethane diisocyanate and any desired mixtures of these isomers (MDI), mixtures of MDI and MDI homologs (polymeric MDI or PMDI), 1,3- and 1,4-phenylene diisocyanate, 2,3,5,6-tetramethyl-1,4-diisocyanatobenzene, naphthalene-1,5-diisocyanate (NDI), 3,3′-dimethyl-4,4′-diisocyanatobiphenyl (TODI), dianisidine diisocyanate (DADI), 1,3,5-tris(isocyanatomethyl)benzene, tris(4-isocyanatophenyl)methane, tris-(4-isocyanatophenyl)thiophosphate, oligomers and polymers of the aforementioned isocyanates, and any desired mixtures of the aforementioned isocyanates. Preference is given to MDI and TDI.

Examples of suitable aliphatic polyisocyanates include monomeric diisocyanates or triisocyanates such as 1,4-tetramethylene diisocyanate, 2-methylpentamethylene 1,5-diisocyanate, 1,6-hexamethylene diisocyanate (HDI), 2,2,4- and 2,4,4-trimethyl-1,6-hexamethylene diisocyanate (TMDI), 1,10-decamethylene diisocyanate, 1,12-dodecamethylene diisocyanate, lysine diisocyanate and lysine ester diisocyanate, cyclohexane-1,3- and 1,4-diisocyanate, 1-methyl-2,4- and 2,6-diisocyanatocyclohexane and any desired mixtures of these isomers (HTDI or H₆TDI), 1-isocyanato-3,3,5-trimethyl-5-isocyanatomethylcyclohexane (=isophorone diisocyanate or IPDI), perhydro-2,4′- and 4,4′-diphenylmethane diisocyanate (HMDI or H₁₂MDI), 1,4-diisocyanato-2,2,6-trimethylcyclohexane (TMCDI), 1,3- and 1,4-bis(isocyanate-methyl)cyclohexane, m- and p-xylylene diisocyanate (m- and p-XDI), m- and p-tetramethyl-1,3- and -1,4-xylylene diisocyanate (m- and p-TMXDI), bis(1-isocyanato-1-methylethyl)naphthalene, dimer and trimer fatty acid isocyanates such as 3,6-bis(9-isocyanatononyl)-4,5-di-(1-heptenyl)cyclohexane (dimeryl diisocyanate), α,α,α′,α′,α″,α″-hexamethyl-1,3,5-mesitylene triisocyanate, oligomers and polymers of the aforementioned isocyanates, and also any desired mixtures of the aforementioned isocyanates. Preference is given to HDI and IPDI.

Preferred polyurethane polymers PUP are those having aromatic isocyanate groups.

In another embodiment a suitable polyisocyanate P is a polyisocyanate PI in the form of a monomeric diisocyanate or triisocyanate or of an oligomer of a monomeric diisocyanate, suitable monomeric diisocyanates or triisocyanates being, for example, the aforementioned aromatic and aliphatic diisocyanates and triisocyanates.

Suitable oligomers of a monomeric diisocyanate include more particularly the oligomers of HDI, IPDI and TDI. In practice, such oligomers usually constitute mixtures of substances having different degrees of oligomerization and/or chemical structures. They preferably have an average NCO functionality of 2.1 to 4.0 and contain, more particularly isocyanurate groups, iminooxadiazinedione groups, uretdione groups, urethane groups, biuret groups, allophanate groups, carbodiimide groups, uretonimine groups or oxadiazinetrione groups. They preferably have a low monomeric diisocyanate content. Commercially available products are, more particularly, HDI biurets, for example Desmodur® N 100 and Desmodur® N 3200 (from Bayer), Tolonate® HDB and Tolonate® HDB-LV (from Rhodia) and also Duranate® 24A-100 (from Asahi Kasai); HDI isocyanurates, examples being Desmodur® N 3300, Desmodur® N 3600 and Desmodur® N 3790 BA (from Bayer), Tolonate® HDT, Tolonate® HDT-LV and Tolonate® HDT-LV2 (from Rhodia), Duranate® TPA-100 and Duranate® THA-100 (from Asahi Kasei) and also Coronate® HX (from Nippon Polyurethane); HDI uretdiones, an example being Desmodur® N 3400 (from Bayer); HDI iminooxadiazinediones, an example being Desmodur® XP 2410 (from Bayer); HDI allophanates, an example being Desmodur® VP LS 2102 (from Bayer); IPDI isocyanurates, examples being Desmodur® Z 4470 (from Bayer) and Vestanat® T1890/100 (from Evonik); TDI oligomers, an example being Desmodur® IL (from Bayer); and also mixed isocyanurates based on TDI/HDI, as for example Desmodur® HL (from Bayer).

In another embodiment the polyisocyanate P is a polyisocyanate PI in the form of a room-temperature-liquid form of MDI or a form of polymeric MDI (PMDI).

Room-temperature-liquid forms of MDI (known as “modified MDI”) constitute mixtures of MDI with MDI derivatives, such as MDI carbodiimides, MDI uretonimines or MDI urethanes, for example. Examples of commercially available modified MDI are products including Desmodur® CD, Desmodur® PF and Desmodur® PC (from Bayer), Lupranat® MM 103 (from BASF), Isonate® M 143 (from Dow) and also Suprasec® 2020 and Suprasec® 2388 (from Huntsman).

Polymeric MDI or PMDI identifies mixtures of MDI and MDI-homologs. Commercially available PMDI products are, for example Desmodur® VL, Desmodur® VL 50, Desmodur® VL R 10, Desmodur® VL R 20 and Desmodur® VKS 20 F (from Bayer), Lupranat® M 10 R and Lupranat® M 20 R (from BASF), Isonate® M 309, Voranate® M 229 and Voranate M® 580 (from Dow) and also Suprasec® 5025, Suprasec® 2050 and Suprasec® 2487 (from Huntsman).

Preferred as polyisocyanate PI are PMDI, room-temperature-liquid forms of MDI, and also oligomers of HDI, IPDI and TDI, more particularly the isocyanurates.

Particularly preferred polyisocyanates PI are those having aromatic isocyanate groups.

Most preferred as polyisocyanate PI are MDI, especially MDI grades with a high fraction, more particularly 50% by weight or more, of 2,4′ isomer, PMDI, and also room-temperature-liquid forms of MDI.

In another embodiment the polyisocyanate P is a mixture composed of at least one polyurethane polymer PUP and at least one polyisocyanate PI, as have been described above.

Typically the polyisocyanate P is present in an amount of 5% to 95% by weight, preferably in an amount of 10% to 90% by weight, based on the overall composition. In filled compositions—that is compositions which comprise a filler—the polyisocyanate P is present preferably in an amount of 5% to 60% by weight, more particularly 10% to 50% by weight, based on the overall composition.

The curable composition described further comprises at least one aldehyde- or ketone-blocked amine BA.

An aldehyde- or ketone-blocked amine BA is a compound which comprises at least one amino group which is blocked by means of aldehyde or ketone, selected from the group consisting of aldimino groups, ketimino groups, enamino groups and oxazolidino groups. Said aldimine or aldehyde or ketone is in this case free from OH, SH and NH groups.

A suitable blocked amine BA in one embodiment is an aldimine BA1 of the formula (I),

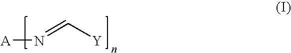

where

n is an integer from 1 to 5

A is the radical of an amine B following removal of n primary amino groups, and

Y is an organic radical having 1 to 35 C atoms and optionally containing heteroatoms.

A “primary amino group” in the present document identifies an NH₂ group which is attached to an organic radical, and a “secondary amino group” identifies an NH group which is attached to two organic radicals which may also together be part of a ring.

Preferred aldimines BA1 of the formula (I) are aldimines BA1 of the formula (I a) and (I b),

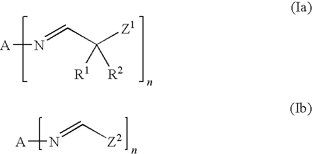

where
R¹ and R² either
independently of one another are each a monovalent hydrocarbon radical having 1 to 12 C atoms,
or together are a divalent hydrocarbon radical having 4 to 20 C atoms which is part of an optionally substituted carbocyclic ring having 5 to 8, preferably 6, C atoms;
   Z¹ is a monovalent hydrocarbon radical having 1 to 32 C atoms which optionally contains at least one heteroatom, more particularly oxygen in the form of ether, carbonyl or ester groups, or more particularly nitrogen in the form of tertiary amino groups;
   Z² either
is a substituted or unsubstituted aryl or heteroaryl group which has a ring size of 5 to 8, preferably 6, atoms,
or is

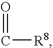

where R⁸ is a hydrogen atom or is an alkoxy group, or is a substituted or unsubstituted alkenyl or arylalkenyl group having at least 6 C atoms;
   and A and n have the definitions already mentioned.

Preferably R¹ and R² in formula (I a) are each a methyl group.

With further preference Z¹ in formula (I a) is a radical of the formula (II) or (III) or (IV),

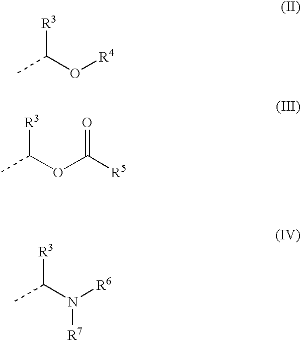

where
R³ is a hydrogen atom or is an alkyl group or is a cycloalkyl group or is an arylalkyl group having 1 to 12 C atoms;
R⁴ is a hydrocarbon radical having 1 to 30 C atoms, which optionally contains ether oxygen atoms;
R⁵ alternatively
is a hydrogen atom,
or is a linear or branched alkyl radical having 1 to 30 C atoms, optionally with cyclic fractions and optionally with at least one heteroatom, more particularly oxygen in the form of ether, carbonyl or ester groups,
or is a singly or multiply unsaturated, linear or branched hydrocarbon radical having 5 to 30 C atoms,
or is an optionally substituted aromatic or heteroaromatic 5- or 6-membered ring;
   R⁶ and R⁷ either
independently of one another are each a monovalent aliphatic, cycloaliphatic or arylaliphatic radical having 1 to 20 C atoms and optionally containing heteroatoms in the form of ether oxygen or tertiary amine nitrogen,
or together are a divalent aliphatic radical having 3 to 20 C atoms, which is part of an optionally substituted heterocyclic ring having 5 to 8, preferably 6, ring atoms and containing optionally, in addition to the nitrogen atom, further heteroatoms in the form of ether oxygen or tertiary amine nitrogen.

Dashed lines in the formulae in this document represent in each case the bond between a substituent and the associated remainder of the molecule.

Preferably R³ in the formulae (II), (III) and (IV) is a hydrogen atom.

Preferably R⁴ in formula (II) is a hydrocarbon radical having 6 to 30, more particularly having 11 to 30, C atoms, which optionally contains ether oxygen atoms.

Preferably R⁵ in formula (III) is a linear or branched alkyl radical having 6 to 30, more particularly having 11 to 30, C atoms, optionally with cyclic fractions and optionally with at least one heteroatom, or is a singly or multiply unsaturated, linear or branched hydrocarbon radical having 6 to 30, more particularly having 11 to 30, C atoms.

Most preferably R⁵ in formula (III) is a C₁₁ alkyl radical.

Preferably R⁶ and R⁷ in formula (IV) are each independently of one another methyl, ethyl, propyl, isopropyl, butyl, 2-ethylhexyl, cyclohexyl or benzyl, or together—with incorporation of the nitrogen atom—they form a ring, more particularly a pyrrolidine, piperidine, morpholine or N-alkylpiperazine ring, this ring being optionally substituted.

An aldimine BA1 of the formula (I) is obtainable by a condensation reaction, with elimination of water, between at least one amine B of the formula (V) and at least one aldehyde ALD of the formula (VI). In such a reaction, the aldehyde ALD of the formula (VI) is used stoichiometrically or in a stoichiometric excess in relation to the amino groups of the amine B.

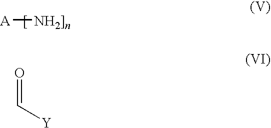

A, n and Y in the formulae (V) and (VI) have the definitions already mentioned.

Suitability as amine B of the formula (V) is possessed in one embodiment by polyamines having at least two primary amino groups, such as, for example
aliphatic, cycloaliphatic or arylaliphatic diamines, examples being ethylenediamine, 1,2-propanediamine, 1,3-propanediamine, 2-methyl-1,2-propanediamine, 2,2-dimethyl-1,3-propanediamine, 1,3-butanediamine, 1,4-butanediamine, 1,3-pentanediamine (DAMP), 1,5-pentanediamine, 1,5-diamino-2-methylpentane (MPMD), 1,6-hexanediamine, 2,5-dimethyl-1,6-hexanediamine, 2,2,4- and 2,4,4-trimethylhexamethylenediamine (TMD), 1,7-heptanediamine, 1,8-octanediamine, 1,9-nonanediamine, 1,10-decanediamine, 1,11-undecanediamine, 1,12-dodecanediamine and methylbis-(3-aminopropyl)amine, 1,2-, 1,3- and 1,4-diaminocyclohexane, bis(4-aminocyclohexyl)methane, bis(4-amino-3-methylcyclohexyl)methane, bis(4-amino-3-ethylcyclohexyl)methane, bis-(4-amino-3,5-dimethylcyclohexyl)methane, bis(4-amino-3-ethyl-5-methylcyclohexyl)methane (M-MECA), 1-amino-3-aminomethyl-3,5,5-trimethylcyclohexane (=isophoronediamine or IPDA), 2- and 4-methyl-1,3-diaminocyclohexane and mixtures thereof, 1,3- and 1,4-bis(aminomethyl)cyclohexane, 2,5(2,6)-bis(aminomethyl)bicyclo[2.2.1]heptane (NBDA), 3(4),8(9)-bis(aminomethyl)tricyclo[5.2.1.0^{2,6}]decane, 1,4-diamino-2,2,6-trimethylcyclohexane (TMCDA), 1,8-menthanediamine, 3,9-bis (3-aminopropyl)-2,4,8,10-tetraoxaspiro[5.5]undecane and 1,3- and 1,4-xylylenediamine;
aliphatic diamines containing ether groups, examples being bis(2-aminoethyl)ether, 3,6-dioxaoctane-1,8-diamine, 4,7-dioxadecane-1,10-diamine, 4,7-dioxadecane-2,9-diamine, 4,9-dioxadodecane-1,12-diamine, 5,8-dioxadodecane-3,10-diamine and higher oligomers of these diamines, bis(3-aminopropyl)polytetrahydrofurans and other polytetrahydrofuran-diamines having molecular weights in the range from, for example, 350 to 5200, and also polyoxyalkylene-diamines. The latter typically represent products from the amination of polyoxyalkylene-diols and are obtainable, for example, under the name Jeffamine® (from Huntsman), under the name Polyetheramine (from BASF) or under the name PC Amine® (from Nitroil). Particularly suitable polyoxyalkylene-diamines are Jeffamine® D-230, Jeffamine® D-400, Jeffamine®D-2000, Jeffamine® D-4000, Jeffamine® XTJ-511, Jeffamine® ED-600, Jeffamine® ED-900, Jeffamine® ED-2003, Jeffamine® XTJ-568, Jeffamine® XTJ-569, Jeffamine® XTJ-523, Jeffamine® XTJ-536, Jeffamine® XTJ-542, Jeffamine® XTJ-559; Polyetheramine D 230, Polyetheramine D 400 and Polyetheramine D 2000, PC Amine® DA 250, PC Amine® DA 400, PC Amine® DA 650, and PC Amine® DA 2000;
aliphatic, cycloaliphatic or arylaliphatic triamines such as 4-aminomethyl-1,8-octanediamine, 1,3,5-tris(aminomethyl)benzene, 1,3,5-tris (aminomethyl)cyclohexane;
polyoxyalkylene-triamines, which typically represent products from the amination of polyoxyalkylene-triols and are obtainable, for example, under the trade name Jeffamine® (from Huntsman), under the name Polyetheramine (from BASF) or under the name PC Amine® (from Nitroil), such as, for example, Jeffamine® T-403, Jeffamine® T-5000; Polyetheramine T403, Polyetheramine T5000; and PC Amine® TA 403, PC Amine® TA 5000;
aromatic diamines and triamines, such as, for example, 1,2-, 1,3- and 1,4-phenylenediamine, 2,4- and 2,6-tolylenediamine (TDA), 3,4-tolylene-diamine, 3,5-dimethylthio-2,4- and -2,6-tolylenediamine, 3,5-diethyl-2,4- and -2,6-tolylenediamine (DETDA), 2,4,6-triethyl-1,3-phenylenediamine, 2,4,6-triisopropyl-1,3-phenylenediamine, 3-ethyl-5-methyl-2,4-tolylenediamine, 3,5-diisopropyl-2,4-tolylenediamine, 3,5-bis(1-methylpropyl)-2,4-tolylenediamine, 3,5-bis(tert-butyl)-2,4-tolylenediamine, 3-ethyl-5-isopropyl-2,4-tolylenediamine, 5-isopropyl-2,4-tolylenediamine, 5-(tert-butyl)-2,4-tolylenediamine, 4,6-bis(1-methylpropyl)-1,3-phenylenediamine, 4-isopropyl-6-(tert-butyl)-1,3-phenylene-diamine, 4-ethyl-6-isopropyl-1,3-phenylenediamine, 4-ethyl-6-(2-methylpropyl)-1,3-phenylenediamine, 4-ethyl-6-(1-methylpropyl)-1,3-phenylenediamine, 4-ethyl-6-(2-methylpropyl)-1,3-phenylenediamine, 4-isopropyl-6-(1-methyl-propyl)-1,3-phenylenediamine, 4-(tert-butyl)-6-(2-methylpropyl)-1,3-phenylene-diamine, 4-cyclopentyl-6-ethyl-1,3-phenylenediamine, 4-cyclopentyl-6-isopropyl-1,3-phenylendiamine, 4,6-dicyclopentyl-1,3-phenylenediamine, 3-isopropyl-2,6-tolylenediamine, 2-methylpropyl 4-chloro-3,5-diaminobenzoate, tert-butyl 4-chloro-3,5-diaminobenzoate, 2,6-diaminopyridine, melamine, 4,4′-, 2,4′- and 2,2′-diaminodiphenylmethane (MDA), 3,3′-dimethyl-4,4′-diamino-diphenylmethane, 3,3′-dichloro-4,4′-diaminodiphenylmethane (MOCA), 3,3′,5,5′-tetraethyl-4,4′-diaminodiphenylmethane (M-DEA), 3,3′,5,5′-tetraethyl-2,2′-dichloro-4,4′-diaminodiphenylmethane (M-CDEA), 3,3′-diisopropyl-5,5′-dimethyl-4,4′-diaminodiphenylmethane (M-MIPA), 3,3′,5,5′-tetraisopropyl-4,4′-diaminodiphenylmethane (M-DIPA), 3,3′,5,5′-tetra-(1-methylpropyl)-4,4′-diaminodiphenylmethane, 3,3′-dimethyl-5,5′-di-tert-butyl-4,4′-diaminodiphenyl-methane, 3,3′-di-tert-butyl-4,4′-diaminodiphenylmethane, 4,4′-diaminodiphenyl sulfone (DDS), 4-amino-N-(4-aminophenyl)benzenesulfonamide, 5,5′-methylenedianthranilic acid, dimethyl 5,5′-methylenedianthranilate, 1,3-propylenebis(4-aminobenzoate), 1,4-butylenebis(4-aminobenzoate), polytetramethylene oxide bis(4-aminobenzoate) (available as Versalink® from Air Products), and 1,2-bis(2-aminophenylthio)ethane.

Preference is given to a polyamine having at least two primary amino groups, selected from the group consisting of 1,6-hexamethylenediamine, MPMD, DAMP, IPDA, TMD, 1,3-xylylenediamine, 1,3-bis(aminomethyl)cyclohexane, bis(4-aminocyclohexyl)methane, bis(4-amino-3-methylcyclohexyl)methane, 3(4),8(9)-bis(aminomethyl)tricyclo[5.2.1.0^{2.6}]decane, 1,2-, 1,3- and 1,4-diaminocyclohexane, 1,4-diamino-2,2,6-trimethyl-cyclohexane, 3,6-dioxaoctane-1,8-diamine, 4,7-dioxadecane-1,10-diamine, 4-aminomethyl-1,8-octanediamine, polyoxyalkylene-polyamines having two or three amino groups, more particularly the D-230, D-400, D-2000, T-403 and T-5000 products available under the trade name Jeffamine® from Huntsman, and analogous compounds from BASF or Nitroil, 1,3- and 1,4-phenylenediamine, 2,4- and 2,6-tolylenediamine, 4,4′-, 2,4′- and 2,2′-diaminodiphenylmethane, 3,3′-dichloro-4,4′-diaminodiphenylmethane, and mixtures of the stated polyamines.

Suitability as amine B of the formula (V) is possessed in another embodiment by amines which contain at least one primary amino group and at least one further reactive group, which is alternatively a hydroxyl group, a secondary amino group or a mercapto group, such as, for example,
amines having one or two primary and one secondary amino group, such as, for example N-methyl-1,2-ethanediamine, N-ethyl-1,2-ethanediamine, N-butyl-1,2-ethanediamine, N-hexyl-1,2-ethanediamine, N-(2-ethylhexyl)-1,2-ethanediamine, N-cyclohexyl-1,2-ethanediamine, 4-aminomethylpiperidine, 3-(4-aminobutyl)piperidine, N-(2-aminoethyl)piperazine, diethylenetriamine (DETA), bishexamethylenetriamine (BHMT), 3-(2-aminoethyl)amino-propylamine; diamines and triamines from the cyanoethylation or cyanobutylation and subsequent hydrogenation of primary monoamines and diamines, examples being N-methyl-1,3-propanediamine, N-ethyl-1,3-propanediamine, N-butyl-1,3-propanediamine, N-hexyl-1,3-propanediamine, N-(2-ethylhexyl)-1,3-propandiamine, N-dodecyl-1,3-propanediamine, N-cyclohexyl-1,3-propanediamine, 3-methylamino-1-pentylamine, 3-ethylamino-1-pentylamine, 3-butylamino-1-pentylamine, 3-hexylamino-1-pentylamine, 3-(2-ethylhexyl)amino-1-pentylamine, 3-dodecylamino-1-pentylamine, 3-cyclohexyl-amino-1-pentylamine, dipropylenetriamine (DPTA), N3-(3-aminopentyl)-1,3-pentanediamine, N5-(3-aminopropyl)-2-methyl-1,5-pentanediamine, N5-(3-amino-1-ethylpropyl)-2-methyl-1,5-pentanediamine, and fatty diamines such as N-cocoalkyl-1,3-propanediamine, N-oleyl-1,3-propanediamine, N-soyaalkyl-1,3-propanediamine and N-tallowalkyl-1,3-propanediamine, or N—(C₁₆₋₂₂ alkyl)-1,3-propanediamine of the kind obtainable for example, under the trade name Duomeen® from Akzo Nobel; the products of the Michael-like addition of aliphatic primary diamines or triamines with acrylonitrile, maleic or fumaric diesters, citraconic diesters, acrylic and methacrylic esters, acrylamides and methacrylamides, and itaconic diesters, reacted in a 1:1 molar ratio;
hydroxy amines having a hydroxyl group and a primary amino group, such as, for example, 2-aminoethanol, 2-methylaminoethanol, 1-amino-2-propanol, 3-amino-1-propanol, 4-amino-1-butanol, 4-amino-2-butanol, 2-amino-2-methylpropanol, 5-amino-1-pentanol, 6-amino-1-hexanol, 7-amino-1-heptanol, 8-amino-1-octanol, 10-amino-1-decanol, 12-amino-1-dodecanol, 4-(2-aminoethyl)-2-hydroxyethylbenzene, 3-aminomethyl-3,5,5-trimethyl-cyclohexanol; derivatives bearing one primary amino group of glycols such as diethylene glycol, dipropylene glycol, dibutylene glycol and higher oligomers and polymers of these glycols, examples being 2-(2-aminoethoxy)ethanol, Methylene glycol monoamine, α-(2-hydroxymethylethyl)-ω-(2-aminomethyl-ethoxy)-poly(oxy(methyl-1,2-ethanediyl)); derivatives, bearing one hydroxyl group and one primary amino group, of polyalkoxylated alcohols having a functionality of three or more; products from the single cyanoethylation and subsequent hydrogenation of glycols, examples being 3-(2-hydroxyethoxy) propylamine, 3-(2-(2-hydroxyethoxy)ethoxy)propylamine and 3-(6-hydroxyhexyloxy)propylamine;
mercapto amines, such as, for example, 2-aminoethanethiol (cysteamine), 3-aminopropanethiol, 4-amino-1-butanethiol, 6-amino-1-hexanethiol, 8-amino-1-octanethiol, 10-amino-1-decanethiol, 12-amino-1-dodecanethiol and amino thio sugars such as 2-amino-2-deoxy-6-thioglucose;
amines having one or more primary and more than one secondary amino group, such as N,N′-bis(3-aminopropyl)ethylenediamine, triethylenetetramine (TETA), tetraethylenepentamine (TEPA), penta-ethylenehexamine and higher homologs of linear polyethylenamines, N,N′-bis (3-aminopropyl)ethylenediamine, products form the multiple cyanoethylation or cyanobutylation and subsequent hydrogenation of primary diamines and polyamines having two or more primary amino groups, such as N,N′-bis-(3-aminopropyl)ethylenediamine, N,N′-bis(3-aminopropyl)-1,4-diaminobutane, N,N′-bis(3-aminopropyl)-2-methyl-1,5-pentanediamine, N,N′-bis(3-amino-1-ethylpropyl)-2-methyl-1,5-pentanediamine, and polyethylenimines with different degrees of polymerization (molar mass range 500 to 1 000 000 g/mol), of the kind obtainable, for example, under the trade name Lupasol® from BASF in pure form or as aqueous solution, these polyethylenimines containing not only primary and secondary but also tertiary amino groups;
hydroxy amines having at least one hydroxyl group, at least one secondary and at least one primary amino group, of the kind obtainable, for example, from the cyanoethylation or cyanobutylation and subsequent hydrogenation of simple aliphatic hydroxy amines, examples being N-hydroxyethyl-1,2-ethanediamine, N-hydroxypropyl-1,2-ethanediamine, N-hydroxyethyl-1,3-propanediamine, N3-hydroxyethyl-1,3-pentanediamine;
hydroxy amines having more than one hydroxyl group and one or more primary amino groups, more particularly derivatives of polyalkoxylated alcohols having a functionality of three or more or of polyalkoxylated polyamines, and also amino sugars, examples being glucosamine or galactosamine.

Preferred among these are amines which are selected from the group consisting of N-methyl-1,2-ethanediamine, N-ethyl-1,2-ethanediamine, N-cyclohexyl-1,2-ethanediamine, N-methyl-1,3-propanediamine, N-ethyl-1,3-propanediamine, N-butyl-1,3-propanediamine, N-cyclohexyl-1,3-propane-diamine, 4-aminomethylpiperidine, 3-(4-aminobutyl)piperidine, DETA, DPTA, BHMT, fatty diamines such as N-cocoalkyl-1,3-propanediamine, N-oleyl-1,3-propanediamine, N-soyaalkyl-1,3-propanediamine and N-tallowalkyl-1,3-propanediamine, 5-amino-1-pentanol, 6-amino-1-hexanol, 4-(2-aminoethyl)-2-hydroxyethylbenzene, 3-aminomethyl-3,5,5-trimethylcyclohexanol, 2-(2-aminoethoxy)ethanol, triethylene glycol monoamine, 3-(2-hydroxyethoxy)propylamine, 3-(2-(2-hydroxyethoxy)ethoxy)propylamine, and 3-(6-hydroxyhexyloxy)-propylamine.

Suitability as aldehyde ALD of the formula (VI) is possessed by primary and secondary aliphatic aldehydes such as propanal, 2-methylpropanal, butanal, 2-methylbutanal, 2-ethylbutanal, pentanal, 2-methylpentanal, 3-methylpentanal, 4-methylpentanal, 2,3-dimethylpentanal, hexanal, 2-ethylhexanal, heptanal, octanal, nonanal, decanal, undecanal, 2-methylundecanal, dodecanal, methoxyacetaldehyde, cyclopropanecarboxaldehyde, cyclopentanecarboxaldehyde, cyclohexane-carboxaldehyde and diphenylacetaldehyde.

Particularly suitable as aldehyde ALD of the formula (VI) are aldehydes which are not enolizable, since such aldehydes, on reaction with primary amines, form aldimino groups which are unable to undergo tautomerization to enamino groups and which therefore represent particularly well-blocked amino groups. Especially tertiary aliphatic aldehydes and also aromatic aldehydes constitute non-enolizable aldehydes.

Particularly suitable as aldehyde ALD of the formula (VI) are tertiary aliphatic aldehydes ALD1 of the formula (VI a),

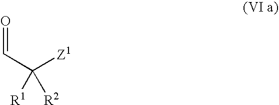

where R¹, R² and Z¹ have the definitions already mentioned.

Examples of suitable aldehydes ALD1 of the formula (VI a) include pivalaldehyde (=2,2-dimethylpropanal), 2,2-dimethylbutanal, 2,2-diethyl-butanal, 1-methylcyclopentanecarboxaldehyde, 1-methylcyclohexane-carboxaldehyde; ethers formed from 2-hydroxy-2-methylpropanal and alcohols such as propanol, isopropanol, butanol and 2-ethylhexanol; esters formed from 2-formyl-2-methylpropionic acid or 3-formyl-3-methylbutyric acid and alcohols such as propanol, isopropanol, butanol and 2-ethylhexanol; esters formed from 2-hydroxy-2-methylpropanal and carboxylic acids such as butyric acid, isobutyric acid and 2-ethylhexanoic acid; and also the ethers and esters, described below as being particularly suitable, of 2,2-disubstituted 3-hydroxypropanals, -butanals or similar higher aldehydes, more particularly of 2,2-dimethyl-3-hydroxypropanal.

Particularly suitable aldehydes ALD1 of the formula (VI a) are, in one embodiment, aldehydes ALD2 of the formula (VI b),

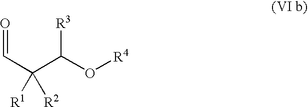

where R¹, R², R³ and R⁴ have the definitions already mentioned.

The aldehydes ALD2 of the formula (VI b) represent ethers of aliphatic, cycloaliphatic or arylaliphatic, 2,2-disubstituted 3-hydroxyaldehydes with alcohols or phenols of the formula R⁴—OH, such as fatty alcohols or phenols, for example. Suitable 2,2-disubstituted 3-hydroxyaldehydes are obtainable in turn from aldol reactions, more particularly crossed aldol reactions, between primary or secondary aliphatic aldehydes, especially formaldehyde, and secondary aliphatic, secondary cycloaliphatic or secondary arylaliphatic aldehydes, such as, for example, isobutyraldehyde, 2-methylbutyraldehyde, 2-ethylbutyraldehyde, 2-methylvaleraldehyde, 2-ethylcaproaldehyde, cyclopentanecarboxaldehyde, cyclohexanecarboxaldehyde, 1,2,3,6-tetrahydro-benzaldehyde, 2-methyl-3-phenylpropionaldehyde, 2-phenylpropionaldehyde (hydratropaldehyde) or diphenylacetaldehyde. Examples of suitable 2,2-disubstituted 3-hydroxyaldehydes are 2,2-dimethyl-3-hydroxypropanal, 2-hydroxymethyl-2-methylbutanal, 2-hydroxymethyl-2-ethylbutanal, 2-hydroxymethyl-2-methylpentanal, 2-hydroxymethyl-2-ethylhexanal, 1-hydroxymethylcyclopentanecarboxaldehyde, 1-hydroxymethylcyclohexane-carboxaldehyde, 1-hydroxymethylcyclohex-3-enecarboxaldehyde, 2-hydroxymethyl-2-methyl-3-phenylpropanal, 3-hydroxy-2-methyl-2-phenyl-propanal, and 3-hydroxy-2,2-diphenylpropanal.

Particularly suitable aldehydes ALD2 of the formula (VI b) are 2,2-dimethyl-3-phenoxypropanal, 3-cyclohexyloxy-2,2-dimethylpropanal, 2,2-dimethyl-3-(2-ethylhexyloxy)propanal, 2,2-dimethyl-3-lauroxypropanal and 2,2-dimethyl-3-stearoxypropanal.

Particularly suitable aldehydes ALD1 of the formula (VI a) are, in a further embodiment, aldehydes ALD3 of the formula (VI c),

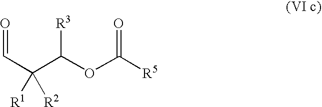

where R¹, R², R³ and R⁵ have the definitions already mentioned.

The aldehydes ALD3 of the formula (VI c) represent esters of the above-described 2,2-disubstituted 3-hydroxyaldehydes, such as 2,2-dimethyl-3-hydroxypropanal, 2-hydroxymethyl-2-methylbutanal, 2-hydroxymethyl-2-ethyl butanal, 2-hydroxymethyl-2-methylpentanal, 2-hydroxymethyl-2-ethyl hexanal, 1-hydroxymethylcyclopentanecarboxaldehyde, 1-hydroxy-methylcyclohexanecarboxaldehyde, 1-hydroxymethylcyclohex-3-enecarboxaldehyde, 2-hydroxymethyl-2-methyl-3-phenylpropanal, 3-hydroxy-2-methyl-2-phenylpropanal, and 3-hydroxy-2,2-diphenylpropanal, for example, with suitable carboxylic acids.

Examples of carboxylic acids suitable for this reaction include saturated aliphatic carboxylic acids, such as formic acid, acetic acid, propionic acid, butyric acid, isobutyric acid, valeric acid, caproic acid, 2-ethylcaproic acid, oenanthic acid, caprylic acid, pelargonic acid, capric acid, undecanoic acid, lauric acid, tridecanoic acid, myristic acid, pentadecanoic acid, palmitic acid, margaric acid, stearic acid, nonadecanoic acid, arachidic acid; monounsaturated aliphatic carboxylic acids such as palmitoleic acid, oleic acid, erucic acid; polyunsaturated aliphatic carboxylic acids such as linoleic acid, linolenic acid, elaeostearic acid, arachidonic acid; cycloaliphatic carboxylic acids such as cyclohexanecarboxylic acid; arylaliphatic carboxylic acids such as phenylacetic acid; aromatic carboxylic acids such as benzoic acid, naphthoic acid, toluic acid, anisic acid; isomers of these acids; fatty acid mixtures from the industrial saponification of natural oils and fats such as, for example, rapeseed oil, sunflower oil, linseed oil, olive oil, coconut oil, oil palm kernel oil, and oil palm oil; and also monoalkyl and monocryl esters of dicarboxylic acids of the kind obtained from the single esterification of dicarboxylic acids such as succinic acid, glutaric acid, adipic acid, pimelic acid, suberic acid, azelaic acid, sebacic acid, 1,12-dodecanedioic acid, maleic acid, fumaric acid, hexahydrophthalic acid, hexahydroisophthalic acid, hexahydroterephthalic acid, 3,6,9-trioxaundecanedioic acid and similar derivatives of polyethylene glycol, with alcohols such as methanol, ethanol, propanol, butanol, higher homologs and isomers of these alcohols. Preferred carboxylic acids are those having at least 7 C atoms, more particularly those having at least 11 C atoms.

Particularly suitable aldehydes ALD1 of the formula (VI a) in another embodiment are aldehydes ALD4 of the formula (VI d),

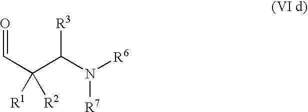

where R¹, R², R³, R⁶ and R⁷ have the definitions already mentioned.

An aldehyde ALD4 of the formula (VI d) is obtainable more particularly as a product of a Mannich reaction or of an α-amino alkylation analogous to the Mannich reaction, of the kind known from the art literature; hence it may also be referred to as a Mannich base. In this case a secondary aldehyde A1, a further aldehyde A2 and a secondary aliphatic amine A3 are reacted with elimination of water to form an aldehyde ALD4.

Examples of suitable aldehydes A1 include isobutyraldehyde, 2-methylbutyraldehyde, 2-ethylbutyraldehyde, 2-methylvaleraldehyde, 2-ethylcaproaldehyde, cyclopentanecarboxaldehyde, cyclohexane-carboxaldehyde, 1,2,3,6-tetrahydrobenzaldehyde, 2-methyl-3-phenyl-propionaldehyde, 2-phenylpropionaldehyde and diphenylacetaldehyde. Isobutyraldehyde is preferred.

Examples of suitable aldehydes A2 include formaldehyde, acetaldehyde, propionaldehyde, butyraldehyde, isobutyraldehyde, phenyl-acetaldehyde, benzaldehyde and substituted benzaldehydes, and also glyoxylic esters, more particularly ethyl glyoxylate. Formaldehyde is preferred.

Examples of suitable secondary aliphatic amines A3 include dimethylamine, diethylamine, dipropylamine, diisopropylamine, dibutylamine, diisobutylamine, di-sec-butylamine, dihexylamine, di(2-ethylhexyl)amine, dicyclohexylamine, N-methylbutylamine, N-ethylbutylamine, N-methyl-cyclohexylamine, N-ethylcyclohexylamine, di-2-methoxyethylamine, pyrrolidine, piperidine, N-methylbenzylamine, N-isopropylbenzylamine, N-tert-butylbenzylamine, dibenzylamine, morpholine, 2,6-dimethylmorpholine, bis(3-dimethylaminopropyl)amine, N-methylpiperazine or N-ethylpiperazine. Preferred of these are dimethylamine, diethylamine, dipropylamine, diisopropylamine, dibutylamine, diisobutylamine, bis(2-ethylhexyl)amine, N-methylcyclohexylamine, N-methylbenzylamine, N-isopropylbenzylamine, N-tert-butylbenzylamine, dibenzylamine, pyrrolidine, piperidine, hexamethylenimine, morpholine and 2,6-dimethylmorpholine.

Particularly suitable as aldehyde ALD of the formula (VI) in a further embodiment are aldehydes ALD6 of the formula (VI e),

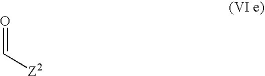

where Z² has the definitions already stated.

Examples of aldehydes ALD5 of this kind include aromatic aldehydes, such as benzaldehyde, 2- and 3- and 4-tolualdehyde, 4-ethyl- and 4-propyl- and 4-isopropyl- and 4-butyl-benzaldehyde, 2,4-dimethylbenzaldehyde, 2,4,5-trimethylbenzaldehyde, 4-acetoxybenzaldehyde, 4-anisaldehyde, 4-ethoxybenzaldehyde, the isomeric di- and trialkoxybenzaldehydes, 2-, 3- and 4-nitrobenzaldehyde, 2- and 3- and 4-formylpyridine, 2-furfuraldehyde, 2-thiophenecarbaldehyde, 1- and 2-naphthylaldehyde, 3- and 4-phenyloxybenzaldehyde, quinoline-2-carbaldehyde and its 3-, 4-, 5-, 6-, 7- and 8-position isomers, and also anthracene-9-carbaldehyde; and also, additionally, glyoxal, glyoxalic esters such as methyl glyoxalate, for example, and cinnamaldehyde and substituted cinnamaldehydes.

Preferred aldehydes ALD of the formula (VI) are the stated aldehydes ALD2 of the formula (VI b), ALD3 of the formula (VI c), ALD4 of the formula (VI d), and ALD5 of the formula (VI e). Particularly preferred of these are the aldehydes ALD3 of the formula (VI c), more particularly those in which the radical R⁵ has 6 to 30 C atoms. The most preferred are odorless aldehydes ALD3 of the formula (VI c), in which the radical R⁵ has 11 to 30 C atoms.

The most preferred aldehyde ALD is 2,2-dimethyl-3-lauroyloxypropanal.

Suitability as blocked amine BA is possessed in one further embodiment by a ketimine BA2 of the formula (VII),

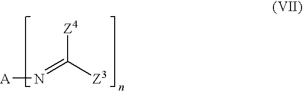

where
Z³ and Z⁴ either
independently of one another are each a monovalent hydrocarbon radical having 1 to 12 C atoms,
or together are a divalent hydrocarbon radical having 4 to 20 C atoms which is part of an optionally substituted carbocyclic ring having 5 to 8, preferably 6, C atoms;
   and n and A have the definitions already mentioned.

A ketamine BA2 of the formula (VII) is obtainable by a condensation reaction, with elimination of water, between at least one amine B of the formula (V) and at least one ketone of the formula (VIII). In such a reaction, the ketone of the formula (VIII) is used stoichiometrically or in a stoichiometric excess in relation to the amino groups of the amine B.

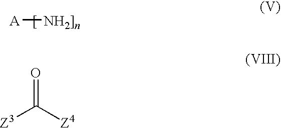

In formula (VIII) Z³ and Z⁴ have the definitions already mentioned.

Particularly suitable as ketone of the formula (VIII) are acetone, methyl ethyl ketone, methyl propyl ketone, methyl isopropyl ketone, methyl isobutyl ketone, methyl pentyl ketone, methyl isopentyl ketone, diethyl ketone, dipropyl ketone, diisopropyl ketone, dibutyl ketone, diisobutyl ketone, cyclopentanone, cyclohexanone and acetophenone.

Suitable amines B of the formula (V) are the amines B already mentioned above.

Examples of suitable commercial diketimines include Epikure® Curing Agent 3502 (from Resolution Performance Products) and Desmophen® LS 2965A (from Bayer).

A suitable blocked amine BA in another embodiment is an enamine BA3, which contains at least one enamino group of the formula (IX), and which is obtainable, for example, from the reaction of at least one secondary amine C with at least one aliphatic or cycloaliphatic aldehyde or ketone of the formula (X) with elimination of water.

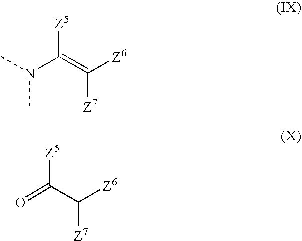

In the formulae (IX) and (X)
Z⁶ and Z⁶ either
independently of one another are each a hydrogen atom or a monovalent hydrocarbon radical having 1 to 12 C atoms,
or together are a divalent hydrocarbon radical having 3 to 20 C atoms, which is part of an optionally substituted carbocyclic ring having 5 to 8, preferably 6, C atoms;
and
Z⁷ is a hydrogen atom or is a monovalent hydrocarbon radical having 1 to 12 C atoms.

A suitable secondary amine C in one embodiment encompasses amines having at least two secondary amino groups, such as, for example, piperazine, tetramethylpiperazine, homopiperazine, 1,3-di(piperidin-4-yl) propane, N,N′-dimethylhexamethylenediamine, and homologs with higher alkyl or cycloalkyl groups instead of the methyl groups, N,N′-dimethyl-diethylenetriamine and N,N′-dimethyldipropylenetriamine.

A suitable secondary amine C in another embodiment encompasses amines having a hydroxyl group and a secondary amino group, such as, for example N-(2-hydroxyethyl)piperazine, 4-hydroxypiperidine and also monoalkoxylated primary monoamines, i.e. reaction products of primary monoamines such as, for example, methylamine, ethylamine, propylamine, butylamine, hexylamine, 2-ethylhexylamine, benzylamine and fatty amines such as laurylamine or stearylamine, with epoxides such as ethylene oxide, propylene oxide or butylene oxide in a stoichiometric ratio of 1:1, examples being N-methylethanolamine, N-ethylethanolamine, N-butylethanolamine and N-butylisopropanolamine.

A suitable secondary amine C in another embodiment encompasses amines having a mercapto group and a secondary amine group, such as N-(2-mercaptoethyl)piperazine, 4-mercapto-piperidine and 2-mercaptoethyl-butylamine.

Suitability as aldehyde or ketone of the formula (X) is possessed by aldehydes which in the α-position to the carbonyl group have at least one hydrogen atom and are therefore enolizable, such as for example, propanal, 2-methylpropanal, butanal, 2-methylbutanal, 2-ethylbutanal, pentanal, 2-methylpentanal, 3-methylpentanal, 4-methylpentanal, 2,3-dimethylpentanal, hexanal, 2-ethylhexanal, heptanal, octanal, nonanal, decanal, undecanal, 2-methylundecanal, dodecanal, methoxyacetaldehyde, cyclopropane-carboxaldehyde, cyclopentanecarboxaldehyde, cyclohexanecarboxaldehyde, and diphenylacetaldehyde; and also by ketones which in the α-position to the carbonyl group have at least one hydrogen atom and are therefore enolizable, such as acetone, methyl ethyl ketone, methyl propyl ketone, methyl isopropyl ketone, methyl isobutyl ketone, methyl pentyl ketone, methyl isopentyl ketone, diethyl ketone, dipropyl ketone, diisopropyl ketone, dibutyl ketone, diisobutyl ketone, and also, in particular cyclic ketones, such as cyclopentanone and cyclohexanone, for example.

A suitable blocked amine BA in another embodiment is an oxazolidine BA4 of the formula (XI),

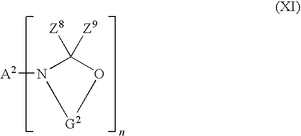

where
A² is the radical of an amine following removal of n secondary amino groups;
G² is an optionally substituted C₂ or C₃ alkylene radical;
Z⁸ and Z⁹ independently of one another are each a hydrogen atom or a monovalent hydrocarbon radical having 1 to 12 C atoms;
and n has the definitions already mentioned.

“Oxazolidino group” in the present document identifies not only tetrahydrooxazole groups (5-membered ring) but also tetrahydrooxazine groups (6-membered ring).

An oxazolidine BA4 of the formula (XI) is obtainable for example from the reaction of at least one amine D of the formula (XII) with at least one aldehyde or ketone of the formula (XIII) with elimination of water.

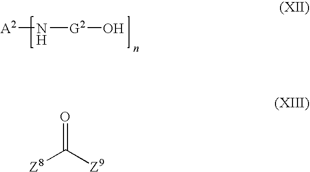

In formulae (XII) and (XIII) A², G², n, Z⁸ and Z⁹ have the definitions already mentioned.

Suitability as amine D of the formula (XII) is possessed by aliphatic hydroxyamines having a secondary amino group, such as diethanolamine, dipropanolamine, and diisopropanolamine, for example.

A preferred amine D is diethanolamine, which can be reacted with an aldehyde or ketone of the formula (XIII) to give an oxazolidine of the formula (XI a).

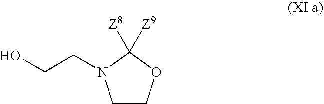

In formula (XI a) Z⁸ and Z⁹ have the definitions already mentioned.

An oxazolidine of the formula (XI a) can be reacted with a polyisocyanate in such a way that the hydroxyl groups react with isocyanate groups. Polyoxazolidines are obtainable in this way.

Suitable aldehydes or ketones of the formula (XIII) are the aldehydes or ketones of the formula (XI), already mentioned, and also, in addition, for example formaldehyde, benzaldehyde and substituted benzaldehydes. 2-Methylpropanal is preferred.

Examples of suitable commercial oxazolidines are curing agent OZ (from Bayer), Zoldine® RD-20, Zoldine® MS-52 and Zoldine® MS Plus (from Angus Chemical), and also Incozol® 2, Incozol® 3, Incozol® LV, Incozol® 4, Incozol® HP and Incozol® NC (from Industrial Copolymers).

Also suitable as blocked amine BA are compounds having two or more of the blocked amino groups described, which are different from one another.

An example of a suitable commercial blocked amine having an aldimino group and an oxazolidino group is Zoldine® RD-4 (from Angus Chemical).

In the formulae (I), (I a), (I b), (VII) and (XI), the index n is preferably 1 or 2 or 3, and blocked amines BA of the formulae (I), (I a), (I b), (VII) and (XI) with the index n=1 contain not only the blocked amino group but preferably at least one reactive group in the form of a hydroxyl group, a secondary amino group or a mercapto group, on the amine moiety.

The blocked amino groups in the form of aldimino groups, ketimino groups, enamino groups and/or oxazolidino groups in the blocked amine BA react extremely slowly or not at all with isocyanate groups in the absence of moisture.

In the curable composition the blocked amine BA is present advantageously in an amount such that the ratio between the number of blocked amino groups and any hydroxyl, mercapto and secondary amino groups present and the number of isocyanate groups, is 0.1 to 1.1, preferably 0.2 to 0.9, more particularly 0.5 to 0.9. If blocked amino groups in the form of oxazolidino groups are present, they are counted twice, since, after hydrolysis has taken place, they are formally difunctional with respect to isocyanate groups.

Preferred as blocked amine BA in the curable composition described are the aldimines BA1 of the formula (I), the ketimines BA2 of the formula (VII), the enamines BA3, containing enamino groups, of the formula (IX) and the oxazolidines BA4 of the formula (XI).

Particularly preferred as blocked amine BA are the aldimines BA1, the ketimines BA2 and the oxazolidines BA4. More particularly preferred are the aldimines BA1 of the formula (I a) and of the formula (I b) and the oxazolidines BA4.

Most preferred as blocked amine BA are the aldimines BA1 of the formula (I a), in which Z¹ is a radical of the formula (II) or (III) or (IV), more particularly a radical of the formula (III).

The curable composition described further comprises at least one hydrazide HY of a carboxylic or sulfonic acid that has a melting point of at least 100° C., more particularly of at least 150° C. The melting point of the hydrazide HY is preferably below 300° C.

In the present document the term “carboxylic acid” also encompasses carbonic acid.

A hydrazide HY of a carboxylic acid has more particularly the formula (XIV a) or (XIV b), while a hydrazide HY of a sulfonic acid has the formula (XV).

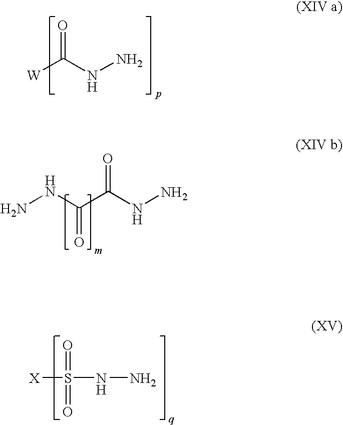

In the formulae (XIV a), (XIV b) and (XV)

W is the p-valent radical of a hydrazide HY of a carboxylic acid, that has a melting point of at least 100° C., more particularly of at least 150° C., following removal of p carboxylic hydrazide groups;

X is the q-valent radical of a hydrazide HY of a sulfonic acid, that has a melting point of at least 100° C., more particularly of at least 150° C., following removal of q sulfonic hydrazide groups;

m is zero or 1;

p is 1 or 2 or 3 or 4, and

g is 1 or 2 or 3 or 4.

Preferably p is 2.

The hydrazide HY is obtainable for example, through the condensation of suitable carboxylic acids or sulfonic acids with hydrazine or hydrazine hydrate. Suitable carboxylic or sulfonic hydrazides having a melting point of more than 100° C. are, for example, the following:

hydrazides of aliphatic and arylaliphatic carboxylic acids such as lauric acid, palmitic acid, stearic acid, cyanacetic acid, 2,4-dichlorophenoxyacetic acid, 4-nitrophenoxyacetic acid, 1-naphthylacetic acid; hydrazides of aromatic and heteroaromatic carboxylic acids such as benzoic acid, 2-, 3- and 4-chlorobenzoic acid, 2-, 3- and 4-bromobenzoic acid, 2- and 4-toluic acid, 2-, 3- and 4-nitrobenzoic acid, salicylic acid, 4-tert-butylbenzoic acid, 4-methoxybenzoic acid, 4-ethoxybenzoic acid, 4-trifluorobenzoic acid, 4-dimethylaminobenzoic acid, the isomeric dichlorobenzoic acids, dimethoxybenzoic acids and trimethoxybenzoic acids, monomethyl terephthalate, 1-naphthylcarboxylic acid, 3-hydroxy-2-naphthylcarboxylic acid, 4-biphenylcarboxylic acid, nicotinic acid, isonicotinic acid, 2-thiophenecarboxylic acid, 4-imidazolecarboxylic acid, 3-pyrazolecarboxylic acid; monohydrazines and dihydrazides of dicarboxylic acids such as oxalic acid, succinic acid, glutaric acid, adipic acid, pimelic acid, suberic acid, azelaic acid, sebacic acid, dodecanedicarboxylic acid, isophthalic acid, terephthalic acid; mono-, di- and trihydrazides of tricarboxylic acids such as benzenetricarboxylic acid; the dihydrazide of carbonic acid (carbodihydrazide); hydrazides of sulfonic acids such as benzenesulfonic acid, 4-toluenesulfonic acid; dihydrazides of disulfonic acids such as propylenedisulfonic acid, butylenedisulfonic acid, o-, m- and p-benzenedisulfonic acid and naphthalenedisulfonic acid.

Additionally suitable as hydrazide HY are cyclic hydrazides of dicarboxylic acids, that represent N-amino imides, examples being N-aminophthalimide, N-aminosuccinimide, 2-amino-3a,4,7,7a-tetrahydro-isoindole-1,3-dione, 4-amino-4-azatricyclo[5.2.1.0^{2,6}]dec-8-ene-3,5-dione, 4-amino-4-aza-10-oxatricyclo[5.2.1.0^{2,6}]dec-8-ene-3,5-dione.

Preferred as hydrazide HY are carboxylic hydrazides.

Particularly preferred as hydrazide HY are carboxylic dihydrazides.

The carboxylic dihydrazide HY is preferably selected from the group consisting of carbodihydrazide, oxalic dihydrazide, succinic dihydrazide, adipic dihydrazide, suberic dihydrazide, azelaic dihydrazide, sebacic dihydrazide, dodecanoic dihydrazide and isophthalic dihydrazide.

The most preferred hydrazide HY is adipic dihydrazide,

Hydrazide groups are able in principle to react both with isocyanate groups and with aldehyde groups and keto groups. The reaction of carboxylic hydrazide groups with isocyanate groups forms acyl semicarbazide groups of the formula (XVI a), whereas the reaction with aldehyde groups or keto groups with elimination of water forms groups of the formula (XVII a). Sulfonic hydrazide groups form groups that are analogous to these of the formula (XVI b) and (XVII b).

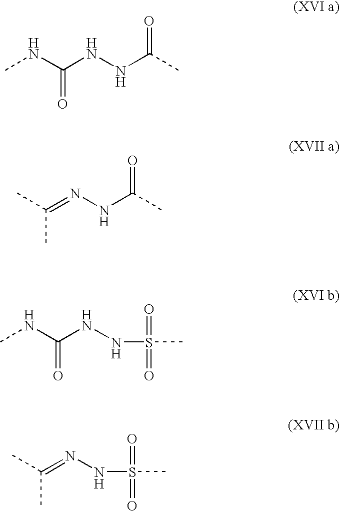

The hydrazide HY is solid at room temperature and has a melting point of at least 100° C., preferably of at least 150° C. Its reactivity toward isocyanate groups and its reactivity toward aldehyde groups and keto groups are greatly restricted at temperatures significantly below its melting point. At room temperature it shows no significant reaction either with isocyanate groups or with aldehyde groups or keto groups. At room temperature or slightly elevated temperature, the hydrazide HY is storage-stable with isocyanate groups. Only at a relatively highly elevated temperature, more particularly at 80° C. and above, do the aforementioned reactions take place to any notable extent.

The hydrazide HY in the curable composition is present in an amount of 0.3 to 1.1 equivalents of hydrazide groups per equivalent of aldehyde groups and keto groups, by means of which the amine BA is blocked. Preferably, the hydrazide HY is present in the curable composition in an amount of 0.5 to 1.0 equivalent, more preferably 0.75 to 1.0 equivalent, of hydrazide groups per equivalent of aldehyde groups or keto groups by means of which the amine BA is blocked.

If the hydrazide HY is present in a smaller amount than 0.3 equivalent of hydrazide groups per equivalent of aldehyde groups and keto groups, by means of which the amine BA is blocked, then the outgassing of aldehyde or ketone is reduced only to a slight extent, owing to the stoichiometry. If the hydrazide HY is present in a higher amount than 1.1 equivalents of hydrazide groups per equivalent of aldehyde groups and keto groups by means of which the amine BA is blocked, then no additional reduction is achieved any longer in the outgassing of aldehyde or ketone. In order to obtain maximum reduction in aldehyde or ketone outgassing, the hydrazide HY is present advantageously in a stoichiometric or near stoichiometric amount relative to the aldehyde groups and keto groups, by means of which the amine BA is blocked.

In addition to at least one polyisocyanate P, at least one aldehyde- or ketone-blocked amine BA and at least one hydrazide HY of a carboxylic or sulfonic acid, the curable composition may comprise further auxiliaries and adjuvants.

The curable composition may take the form of a one-pack composition or the form of a two-pack composition.

A “one-pack composition” in the present document identifies a curable composition whose constituents are stored in mixed form in the same container and which is stable in storage at room temperature for a relatively long time period, hence not altering, or not substantially altering its application properties or service properties as a result of storage, and which, following application, cures by exposure to moisture.

A “two-pack composition” in the present document identifies a curable composition whose constituents are present in two different components which are stored in separate containers from one another and are each stable in storage per se. Not until shortly before or during the application of the composition are the two components mixed with one another, whereupon the mixed composition cures, the curing proceeding or being completed, in certain circumstances, only as a result of exposure to moisture.

One-pack compositions have the advantage that they can be applied without a mixing operation while two-pack compositions have the advantage that they cure more rapidly and may include constituents which are not storable together with isocyanates.

In one embodiment the curable composition takes the form of a one-pack composition.

Preferred as polyisocyanate P in the one-pack composition is a polyurethane polymer PUP, or a mixture of a polyurethane polymer PUP and a polyisocyanate PI, as described above.

A preferred blocked amine BA in the one-pack composition is an aldimine BA1 of the formula (I a), more particularly with a radical Z¹ of the formula (II) or (III) or (IV), or an aldimine BA1 of the formula (I b), or an oxazolidine BA4 of the formula (XI).

Where the blocked amine BA has, on the amine moiety, groups that are reactive toward isocyanates such as hydroxyl groups, mercapto groups or non-blocked amino groups, the number of such groups per molecule is preferably one. Blocked amines BA having isocyanate-reactive groups react on mixing with the polyisocyanate P, by forming adducts.

Preferably, however, the blocked amine BA contains no isocyanate-reactive groups such as hydroxyl groups, mercapto groups or non-blocked amino groups.

Examples of auxiliaries and additives suitable for a one-pack composition including the following substances:
plasticizers, examples being carboxylic esters such as phthalates, for example dioctyl phthalate, diisononyl phthalate or diisodecyl phthalate, adipates, for example dioctyl adipate, azelates and sebacates, organic phosphoric and sulfonic esters or polybutenes;
nonreactive thermoplastic polymers, such as, for example homopolymers or copolymers of unsaturated monomers, more particularly from the group encompassing ethylene, propylene, butylene, isobutylene, isoprene, vinyl acetate and alkyl (meth)acrylates, more particularly polyethylenes (PE), polypropylenes (PP), polyisobutylenes, ethylene-vinyl acetate copolymers (EVA) and atactic poly-α-olefins (APAO);
solvents;
inorganic and organic fillers, examples being ground or precipitated calcium carbonates which, if desired, are coated with fatty acids, more particularly stearates; barytes (BaSO₄, also called heavy spar), finely-ground quartzes, calcined kaolins, aluminum oxides, aluminum hydroxides, silicas, more particularly highly disperse silicas from pyrolysis operations, carbon blacks, more particularly industrially produced carbon blacks (referred to below as “carbon black”), PVC powders or hollow beads;
fibers, of polyethylene, for example;
pigments, such as titanium dioxide or iron oxides, for example;
catalysts which accelerate the hydrolysis of the blocked amino groups, more particularly acids or compounds which can be hydrolyzed to acids, examples being organic carboxylic acids such as benzoic acid, salicylic acid or 2-nitrobenzoic acid, organic carboxylic anhydrides such as phthalic anhydride, hexahydrophthalic anhydride and hexahydromethylphthalic anhydride, silyl esters of organic carboxylic acids, organic sulfonic acids such as methanesulfonic acid, p-toluenesulfonic acid or 4-dodecylbenzenesulfonic acid, sulfonic esters, other organic or inorganic acids, or mixtures of the aforementioned acids and acid esters;
catalysts which accelerate the reaction of the isocyanate groups with water, especially metal compounds, examples being organotin compounds such as dibutyltin diacetate, dibut yltin dilaurate, dibutyltin dichloride, dibutyltin diacetylacetonate and dioctyltin dilaurate, bismuth compounds such as bismuth trioctoate and bismuth tris(neodecanoate), and compounds containing tertiary amino groups, such as 2,2′-dimorpholinodiethyl ether and 1,4-diazabicyclo[2.2.2]octane;
rheology modifiers such as, for example, thickeners or thixotropic agents, examples being urea compounds, polyamide waxes, bentonites or fumed silicas;
reactive diluents and crosslinkers, examples being monomeric diisocyanates and also oligomers and derivatives of these diisocyanates, adducts of monomeric diisocyanates with short-chain polyols;
driers, such as, for example molecular sieves, calcium oxide, highly reactive isocyanates such as p-tosyl isocyanate, orthoformic esters, tetraalkoxysilanes such as tetraethoxysilane; trialkoxysilanes, such as methyltrimethoxysilane, isooctyltrimethoxysilane and vinyltrimethoxysilane;
adhesion promoters, more particularly organoalkoxysilanes, also referred to below as “silanes”, examples being epoxysilanes, such as 3-glycidyloxypropyltrimethoxysilane and 3-glycidyloxypropyltriethoxysilane, (meth)acrylosilanes such as 3-methacryloyloxypropyltrimethoxysilane, isocyanatosilanes, such as 3-isocyanatopropyltrimethoxysilane, S-(alkylcarbonyl)mercaptosilanes, such as S-octanoyl-3-mercaptopropyl-triethoxysilane, and aldiminosilanes, such as N-benzylidene-3-aminopropyltrimethoxysilane, and also oligomeric forms of these silanes;
stabilizers against heat, light and UV radiation;
flame retardants;
surface-active substances such as for example wetting agents, flow control agents, de-aerating agents or defoamers;
biocides such as for example algicides, fungicides or fungal growth inhibitors.

It is advantageous to ensure that such additions do not adversely affect the stability of the composition in storage. This means that these additions shall not give rise to any significant extent, in the course of storage, to the reactions that lead to crosslinking, such as hydrolysis of the blocked amino groups or crosslinking of the isocyanate groups. More particularly this implies that all of these additions should not contain any water, or at most only traces of water. It may therefore be sensible to carry out chemical or physical drying of certain additions prior to their incorporation into the composition by mixing.

The one-pack composition preferably comprises at least one catalyst. More particularly, the composition comprises as catalyst a carboxylic acid such as benzoic acid or salicylic acid and/or a tin compound and/or a bismuth compound. It may be advantageous if different catalysts, or different types of catalyst, such as an acid and a metal compound, for example, are mixed with one another.

With further preference the one-pack composition comprises at least one further auxiliary and adjuvant, more particularly selected from the group encompassing plasticizers, fillers and thickeners.

The one-pack composition described is preferably prepared in the absence of moisture and stored at room temperature or slightly elevated temperature. In a suitable climatically impervious packaging or facility, such as a drum, a pouch or a cartridge, for example, its stability in storage is good. The terms “storage-stable” and “stability in storage” in connection with a curable composition refer in the present document to the state in which the viscosity of the composition, at a given application temperature and when stored appropriately, does not, within the period of time under consideration, increase at all, or increases only to such an extent that the composition remains suitable for use in the manner envisaged.

In a further embodiment the curable composition takes the form of a two-pack composition. The two-pack composition is composed of a component K1 and a component K2, which are stored separately from one another and are not mixed with one another until shortly before application.

In one embodiment of the two-pack composition the polyisocyanate P and the blocked amine BA are part of component K1, while component K2 comprises compounds that are reactive toward isocyanate groups, more particularly water and/or polyols and/or polyamines and/or amino alcohols and/or polythiols, and the hydrazide HY is present either in component K1 or in component K2 or in both components.

In another embodiment of the two-pack composition, the polyisocyanate P is part of component K1, while component K2 comprises the blocked amine BA and also compounds that are reactive toward isocyanate groups, more particularly water and/or polyols and/or polyamines and/or amino alcohols and/or polythiols, and the hydrazide HY is present either in component K1 or in component K2 or in both components.

Component K2 preferably comprises at least one blocked amine BA and water.

The hydrazide HY is preferably a constituent of component K2.

Preference as polyisocyanate P in the two-pack composition is given to a polyisocyanate PI, or to a mixture of a polyurethane polymer PUP and a polyisocyanate PI, as have been described above.

Suitable polyols in component K2 are the same commercially customary polyols already mentioned as suitable for preparing a polyurethane polymer PUP, and also those low-molecular-mass, dihydric or polyhydric alcohols said above to be suitable for accompanying use in the preparation of a polyurethane polymer PUP. Suitable polyamines in component K2 are commercially customary aliphatic or aromatic polyamines having primary and/or secondary amino groups, of the type typically used in two-pack polyurethane compositions, such as, for example, 1,5-diamino-2-methylpentane (MPMD), 1,3-xylylenediamine (MXDA), N,N′-dibutylethylenediamine, 3,5-diethyl-2,4(6)-diaminotoluene (DETDA), 3,5-dimethylthio-2,4(6)-diaminotoluene (available, for example, as Ethacure® 300 from Albemarle) and also primary and secondary polyoxyalkylene-diamines, of the type obtainable, for example, under the name Jeffamine® (from Huntsman). Suitable amino alcohols in component K2 are compounds which contain at least one primary or secondary amino group and at least one hydroxyl group such as, for example, 2-aminoethanol, 2-methylaminoethanol, 1-amino-2-propanol and diethanolamine. Examples of suitable polythiols in component K2 are the liquid, mercapto-terminated polymers known under the brand name Thiokol®, and also polyesters of thiocarboxylic acids.

In addition, both components may comprise further auxiliaries and adjuvants as have already been mentioned above for a one-pack composition. In the case of component K2, however, further auxiliaries and adjuvants are additionally also possible. More particularly, these are those assistants and additives which are storable only for a short period, if at all, with aromatic isocyanate groups. In particular, these are catalysts such as:

compounds of zinc, manganese, iron, chromium, cobalt, copper, nickel, molybdenum, lead, cadmium, mercury, antimony, vanadium, titanium, zirconium or potassium, such as zinc(II) acetate, zinc(II) 2-ethylhexanoate, zinc(II) laurate, zinc(II) oleate, zinc(II) naphthenate, zinc(II) acetylacetonate, zinc(II) salicylate, manganese(II) 2-ethylhexanoate, iron(III) 2-ethylhexanoate, iron(III) acetylacetonate, chromium(III) 2-ethylhexanoate, cobalt(II) naphthenate, cobalt(II) 2-ethylhexanoate, copper(II) 2-ethylhexanoate, nickel(II) naphthenate, phenylmercuric neodecanoate, lead(II) acetate, lead(II) 2-ethylhexanoate, lead(II) neodecanoate, lead(II) acetylacetonate, aluminum lactate, aluminum oleate, aluminum(III) acetylacetonate, diisopropoxytitanium bis(ethylacetoacetate), dibutoxytitanium bis(ethylacetoacetate), di-butoxytitanium bis(acetylacetonate), potassium acetate, potassium octanoate; tertiary amines, such as triethylamine, tributylamine, N-ethyldiisopropylamine, N,N,N′,N′-tetramethylethylenediamine, pentamethyldiethylenetriamine and higher homologs thereof, N,N,N′,N′-tetramethylpropylenediamine, pentamethyldipropylenetriamine and higher homologs thereof, N,N,N′,N′-tetramethyl-1,3-butanediamine, N,N,N′N′-tetramethyl-1,6-hexanediamine, bis(dimethylamino)methane, N,N-dimethylbenzylamine, N,N-dimethylcyclohexylamine, N-methyldicyclohexylamine, N,N-dimethylhexadecylamine, bis(N,N-diethylaminoethyl)adipate, N,N-dimethyl-2-phenylethylamine, tris(3-dimethylaminopropyl)amine, 1,4-diazabicyclo[2.2.2]octane (DABCO), 1,8-diazabicyclo[5.4.0]undec-7-ene (DBU), 1,5-diazabicyclo[4.3.0]non-5-ene (DBN) N-methylmorpholine, N-ethylmorpholine, N-cocomorpholine, N,N′-dimethylpiperazine, N-methyl-N′-dimethylaminoethylpiperazine, bis(dimethylaminoethyl)piperazine, 1,3,5-tris(dimethylaminopropyl)hexahydrotriazine, bis(2-dimethylaminoethyl)ether; aromatic nitrogen compounds, such as 4-dimethylaminopyridine, N-methylimidazole, N-vinylimidazole or 1,2-dimethylimidazole; amidines and guanidines, such as 1,1,3,3-tetramethylguanidine; tertiary amines containing active hydrogen atoms, such as triethanolamine, triisopropanolamine, N-methyldiethanolamine, N,N-dimethylethanolamine, 3-(dimethyl-amino)propyldiisopropanolamine, bis(3-(dimethylamino)propyl)iso-propanolamine, bis(3-dimethylaminopropyl)amine, 3-(dimethylamino) propylurea, Mannich bases, such as 2,4,6-tris(dimethylaminomethyl)phenol or 2,4,6-tris(3-(dimethylamino)propylaminomethyl)phenol, N-hydroxypropyl-imidazole, N-(3-aminopropyl)imidazole, and alkoxylation and polyalkoxylation products of these compounds, as for example dimethylaminoethoxyethanol; organic ammonium compounds, such as benzyltrimethylammonium hydroxide, or alkoxylated tertiary amines; so-called “delayed action” catalysts, which are modifications of known metal or amine catalysts, such as reaction products of tertiary amines and carboxylic acids or phenols, for example of 1,4-diazabicyclo[2.2.2]octane or DBU and formic acid or acetic acid; and combinations of the compounds stated, especially of metal compounds and tertiary amines.

The two-pack composition preferably comprises at least one catalyst. More particularly, the composition comprises as catalyst a carboxylic acid such as benzoic acid or salicylic acid and/or a tin compound and/or a bismuth compound. It may be advantageous if different catalysts, or different kinds of catalysts such as an acid and a metal compound, for example, are mixed with one another.

With further preference the two-pack composition comprises at least one further auxiliary and adjuvant, more particularly selected from the group encompassing plasticizers, crosslinkers, fillers and thickeners.

Component K2 preferably contains no isocyanate groups.

The components K1 and K2 described are prepared separately from one another, and at least the component K1 in the absence of moisture. The two components K1 and K2 are stable in storage separately from one another at room temperature or slightly elevated temperature. That is, they can each be kept in a suitable packaging or facility, as, for example, in a drum, a hobbock, a pouch, a bucket or a cartridge, for a period of several months up to a year or more prior to their application, without undergoing alteration in their respective properties to any extent relevant for their utility.

The mixing ratio between the components K1 and K2 is preferably selected such that the groups of components K1 and K2 that are reactive toward isocyanate groups are in an appropriate proportion to the isocyanate groups of component K1. In the two-pack composition described, there are suitably, prior to curing 0.1 to 1.1, preferably 0.5 to 0.95, more preferably 0.6 to 0.95, equivalent(s) of the sum of isocyanate-reactive groups present per equivalent of isocyanate groups, the blocked amino groups being counted with the isocyanate-reactive groups, and water being considered not to belong to the isocyanate-reactive groups. Excess isocyanate groups react in particular directly with water, as for example with atmospheric humidity.

The curable composition described is applied suitably at a temperature below 40° C. It is clear here to the person skilled in the art that the application temperature of the composition is situated, advantageously, well below the melting point of the hydrazide HY present in order to prevent any premature reaction of the hydrazide HY with the isocyanate groups present. The application takes place by the composition being contacted with the surface of a solid, if desired using a suitable auxiliary means. Where the composition is in a paste-like form, application may take place, for example from commercial cartridges, which for relatively small applications are preferably operated manually. Likewise possible is application by means of compressed air from a cartridge or from a drum or hobbock by means of a conveying pump or an extruder, if desired by means of an application robot. Such forms of application are preferred more particularly in applications in industrial manufacturing or in large-scale applications. In the case of a two-pack composition, the components K1 and K2 are mixed with one another by means of a suitable method prior to or during application. Mixing may take place continuously or batchwise. If mixing takes place prior to application, it must be ensured that the time which elapses between the mixing of components K1 and K2 and the application is not too great, since otherwise there may be defects, such as a delayed or incomplete development of adhesion to the surface of the solid, for example.

During and after application, the composition begins to cure. The curable composition reacts with water or moisture and is crosslinked as a result. If sufficient water is present to react a large part or all of the isocyanate groups, the product is a cured composition which has good mechanical properties. The composition can therefore be identified as “moisture-curing”. This operation—also identified as “curing” or “crosslinking”—takes place suitably at room temperature or slightly elevated temperature, more particularly at below 40° C.

The blocked amine BA begins to react with isocyanate groups present, with release of aldehydes and/or ketones, as soon as it comes into contact with water. On ingress of moisture, or of water, aldimino groups present may hydrolyze via intermediate stages to form primary amino groups, the corresponding aldehyde being released. Since this hydroloysis reaction is reversible and the chemical equilibrium is situated significantly on the aldimine side, it can be assumed that, in the absence of amine-reactive groups, only some of the aldimino groups are hydrolyzed. In the presence of isocyanate groups, there is a shift in the hydrolysis equilibrium, since the hydrolyzing aldimino groups react irreversibly with the isocyanate groups to form urea groups. The reaction of the hydrolyzing aldimino groups with isocyanate groups need not necessarily proceed via free amino groups. Also possible of course are reactions with intermediates of the hydrolysis reaction. It is conceivable, for example, for a hydrolyzing aldimino group in the form of a hemiaminal group to react directly with an isocyanate group. On ingress of moisture, ketimino groups present may undergo hydrolysis via intermediate stages to form primary amino groups, with the corresponding ketone being released. In the presence of isocyanate groups, urea groups are likewise formed in this reaction. Enamino groups that are present also react, on ingress of moisture, with isocyanate groups to form urea groups, with release of the corresponding aldehyde or ketone. In the case of the hydrolysis of oxazolidino groups that are present, one secondary amino group and one hydroxyl group, formally, form per oxazolidino group, with release of an aldehyde or ketone. In the presence of isocyanate groups, the secondary amino groups react to give urea groups, and the hydroxyl groups to give urethane groups. In the context of their hydrolysis, therefore, oxazolidino groups are formally difunctional with respect to isocyanate groups. The water that is needed for the composition to cure is either already present in the applied composition, having been for example—in the case of a two-pack composition—a constituent of component K2, or was added to the composition shortly before or during application, or the water diffuses into the composition in the form of atmospheric moisture. In the latter case, the reaction of the blocked amine BA with the isocyanate groups takes place from the outside in, in parallel with the penetration of the atmospheric moisture into the composition. For the case of a two-pack composition which contains hydroxyl groups, mercapto groups or primary or secondary amino groups, these groups likewise react with isocyanate groups present. Excess isocyanate groups react in particular directly with water. As a result of these reactions, the mixed composition crosslinks and ultimately cures to form a solid material.

Curing takes place generally without bubbles, and also in particular with a high cure rate.

The cure rate can be influenced by the nature and amount of one or more catalysts that may be present and/or via the atmospheric moisture, and/or if desired, via the amount of water introduced by way of a component K2.

Curing takes place suitably at room temperature or slightly elevated temperature, more particularly at below 40° C. Under these conditions, on the basis of its high melting point, the hydrazide HY does not react to any significant extent with isocyanates, but instead remains largely unreacted in the cured composition.

In the event that relatively low-volatility aldehydes or ketones are released from the blocked amine BA when the composition cures, more particularly the odorless aldehydes ALD2 of the formula (VI b), and/or ALD3 of the formula (VI c), with the radicals R⁴ or R⁵ having 11 or more C atoms, then these products, under the stated conditions, likewise remain largely in the composition after curing.

When the cured composition is heated, more particularly to 80° C. and higher, the hydrazide HY begins to react with the aldehydes and/or ketones that are present, more particularly with the aldehydes and/or ketones that are released in the hydrolysis of the blocked amine BA. The condensation products that are formed in the reaction between hydrazides and aldehydes and/or ketones, and which are also referred to as hydrazones, are identified in the present document as “aldazides” and as “ketazides” respectively.

Heating of the cured composition may take place, for example, in the course of the normal use of the cured composition, as a result, for example, of a high level of heating, by solar irradiation, for example, of an automobile whose front windshield has been bonded by means of the composition described.

Alternatively, the heating of the cured composition can be brought about deliberately, by heating, for example, the aforementioned automobile—or an installation component thereof that comprises the bonded front windshield—deliberately to 80° C., for example, such that aldehydes and/or ketones present react with the hydrazide HY. Hence the deliberate heating may represent an operating step in the production process.

Given suitable application temperature and curing temperature, the curable composition described is hardly different at all in its curing behavior and its mechanical properties from corresponding compositions without hydrazide HY. The isocyanate groups react, as already described, with the hydrolyzing blocked amino groups of the blocked amine BA and also with any hydroxyl groups, mercapto groups and non-blocked amino groups that may be present, and finally, with water, whereas the hydrazide HY, on the basis of its high melting point, does not participate to any notable extent in the curing reaction. In the cured state, however, the curable composition described exhibits a significantly reduced outgassing, particularly at elevated temperature. Emissions caused by the outgassing of aldehydes or ketones may have disruptive consequences, particularly in interior spaces, by causing, for example, an unpleasant odor, or by leading to instances of irritation of the skin or respiratory tract or by forming films on surfaces. In vehicles, such films may lead to clouding of the glass, which is also referred to as “fogging” (and may be determined as described in DIN 75201). In the automobile industry in particular, there are often limit values for the volatile fractions that escape by outgassing from the materials used in the vehicle interior, such as adhesives, for example.

When the cured composition is heated, more particularly to 80° C. and higher, the hydrazide begins to react with the aldehydes and/or ketones that are released during the hydrolysis of the blocked amine BA. The aldazides and/or ketazides that are formed in such reactions are of sufficiently low volatility not to outgas even at relatively high temperatures.

Consequently, the described hydrazide HY can be used for reducing the outgassing of aldehyde or ketone from the cured composition.

The preferred aldimines BA1 of the formula (I a), in which Z¹ is a radical of the formula (II) or (III) or (IV), release relatively low-volatility aldehydes ALD2 of the formula (VI b) or ALD3 of the formula (VI c) or ALD4 of the formula (VI d) in the course of the curing reaction. The particularly preferred odorless aldehydes ALD2 or ALD3 in particular, in which R⁴ in formula (VI b) or R⁵ in formula (VI c) has 11 to 30 C atoms, remain in the cured composition for a very long time at room temperature, on account of their low volatility. In the absence of a hydrazide HY, aldehydes of this kind gradually escape from the cured composition at elevated temperature, more particularly at temperatures of 80° C. and higher. In the presence of a hydrazide HY, in contrast, such aldehydes can still be effectively bound when the composition is heated, even when this takes place a very long time after curing. The outgassing of such aldehydes from the compositions described is very greatly reduced as a result of the extremely low volatility of the corresponding aldazides, even at significantly increased temperatures as, for example, at 100° C. or more; this is accompanied by a significantly reduced weight loss on the part of the composition. A virtually stoichiometric hydrazide group content in relation to aldehyde groups and keto groups produces a particularly significant reduction in the weight loss of the cured composition.

The hydrazide HY has, as already mentioned, a melting point of at least 100° C., more particularly of at least 150° C. Too low a melting point on the part of the hydrazide used may result in a premature reaction with isocyanate groups, possibly leading to incomplete curing of the composition and/or to inadequate stability in storage of the hydrazide together with isocyanate groups. The stability in storage of a one-pack composition correlates with the melting point of the hydrazide present. Whereas a one-pack composition comprising benzhydrazide, with a melting point of approximately 112° C., is stable in storage at room temperature or not more than slightly elevated temperature, a similar composition comprising adipic dihydrazide, with a melting point of approximately 180° C., has an excellent stability in storage even at a storage temperature of 60° C. A one-pack composition comprising, for example, acetic hydrazide with a melting point of approximately 63° C., i.e. below 100° C., in contrast, is unstable in storage even at room temperature.

A curable composition which, in addition to at least one polyisocyanate P, comprises as hydrazide HY the particularly preferred adipic dihydrazide in the quantity already stated, and comprises, as blocked amine BA, an aldimine BA1 of the formula (I a) with Z¹ of the formula (II) or (III) with radicals R⁴ or R⁵ having 11 to 30 C atoms, has particularly advantageous properties. It exhibits very good stability in storage, cures rapidly and odorlessly at room temperature or slightly elevated temperature, and in the cured state, possesses good mechanical properties. At elevated temperature it exhibits surprisingly low aldehyde outgassing; its weight loss at 80° C. is very low as compared with that of a corresponding composition without hydrazide HY.

It has further been found that the curable composition described, particularly when it comprises aromatic isocyanate groups or reaction products thereof, displays a distinctly reduced propensity toward yellowing, as compared with corresponding compositions without hydrazide HY. The lower yellowing propensity is manifested in particular in the cured composition, more particularly when said composition is heated for a number of hours or days, at temperatures, for example, of 80 to 130° C.

Accordingly, an as-described hydrazide HY or an aldazide formed therefrom can be used for reducing the yellowing of a cured polyurethane composition.

Preferred applications of the curable composition described, and more particularly of its preferred embodiments, are adhesives, sealants, casting compounds, coatings, floor coverings, paints, varnishes, primers or foams.

With their low outgassing behavior, the curable compositions described are particularly suitable as one-pack or two-pack elastic adhesives in vehicle construction, more particularly as glazing adhesives, where exacting requirements are imposed on the materials employed in respect of outgassing, or fogging.

With their relatively low yellowing propensity, moreover, the curable compositions described are particularly suitable as one-pack or two-pack floor coverings.

In a further aspect, the present invention provides a method of adhesively bonding a substrate S1 to a substrate S2, which comprises the steps of:
i) applying an above-described curable composition to a substrate S1 at a temperature below 40° C.;
ii) contacting the applied composition with a substrate S2 within the open time of the composition;
iii) curing the applied composition at a temperature below 40° C.;

or
i′) applying an above-described curable composition to a substrate S1 and to a substrate S2 at a temperature below 40° C.;
ii′) contacting the applied composition with one another within the open time of the composition;
iii′) curing the applied composition at a temperature below 40° C.;
the substrate S2 being composed of the same material as or a different material from the substrate S1.

In a further aspect, the present invention provides a method of sealing. Said method comprises the steps of
i″) applying an above-described curable composition at a temperature below 40° C. between a substrate S1 and a substrate S2, so that the composition is in contact with the substrate S1 and the substrate S2;
ii″) curing the applied composition at a temperature below 40° C.;
the substrate S2 being composed of the same material as or a different material from the substrate S1.

The composition is typically pressed into a joint.

In a further aspect, the present invention provides a method of coating a substrate S1. Said method comprises the steps of:
i′″) applying an above-described curable composition at a temperature below 40° C. to a substrate S1 within the open time of the composition;
ii′″) curing the applied composition at a temperature below 40° C.

In these three methods, suitable substrates S1 and/or S2 are, for example, inorganic substrates such as glass, glass ceramic, concrete, mortar, brick, tile, plaster, and natural stones such as granite or marble; metals or alloys such as aluminum, steel, nonferrous metals, galvanized metals; organic substrates such as leather, fabrics, paper, wood, resin-bound wood-based materials, resin-textile composites, plastics such as polyvinyl chloride (unplasticized and plasticized PVC), acrylonitrile-butadiene-styrene copolymers (ABS), SMC (sheet molding composites), polycarbonate (PC), polyamide (PA), polyesters, PMMA, polyesters, epoxy resins, polyurethanes (PUR), polyoxymethylene (POM), polyolefins (PO), especially polyethylene (PE) or polypropylene (PP) surface-treated by plasma, corona or flames, ethylene/propylene copolymers (EPM) and ethylene/propylene-diene terpolymers (EPDM); coated substrates such as powder-coated metals or alloys; and also inks and paints, more particularly automotive paints.

The substrates may where necessary be pretreated prior to application of the composition. Such pretreatments include in particular, physical and/or chemical cleaning processes, such as abrading, sandblasting, brushing or the like, for example, or treatment with cleaners or solvents or the application of an adhesion promoter, an adhesion-promoter solution or a primer.

In the case of a two-pack composition, the two components K1 and K2 are mixed with one another shortly before application.

The above-described methods of adhesive bonding, sealing and coating—or the use of one of the described curable compositions as an adhesive, sealant, casting compound, coating, floor covering, paint, varnish, primer or foam—produce an article.

Said article is in more particularly a built structure, more particularly a built structure from the construction or civil engineering sectors, or an industrial product or a consumer product, more particularly a window, a household appliance, or a means of transport, more particularly a water or land vehicle, preferably an automobile, a bus, a truck, a train or a boat, or a part for installation in or on a means of transport or an article from the furniture, textile or packaging industry.

The present invention further provides a cured composition AZ, obtained by the curing of the above-described curable composition at a temperature below 40° C. by means of exposure to water, in the form, for example, of atmospheric humidity, and subsequent heating of the resultant cured composition to a temperature of 80° C. or higher, said cured composition comprising at least one compound of the formula (XVIII a) or (XVIII b) or (XIX),

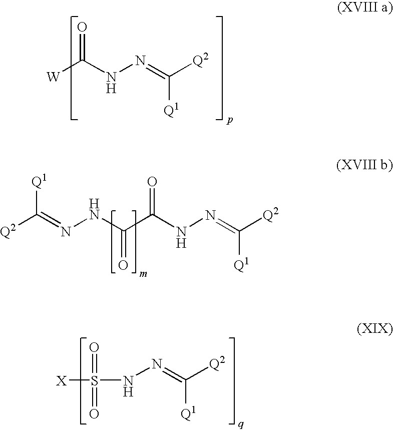

where Q¹ is a hydrogen atom or is Z³ or Z⁵ or Z⁸, and
if Q¹ is a hydrogen atom, Q² is Y,

or
if Q¹ is Z³, Q² is Z⁴,

or
if Q¹ is Z⁵, Q² is

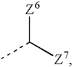

or
if Q¹ is Z⁸, Q² is Z⁹.
   W, X, m, p, q, Y, Z³, Z⁴, Z⁵, Z⁵, Z⁶, Z⁷, Z⁸ and Z⁹ have the definitions already described.

The compounds of the formula (XVIII a), (XVIII b) and (XIX) represent aldazides or ketazides of carboxylic or sulfonic hydrazides. The compounds of the formula (XVIII a) and (XVIII b) are formed by the reaction of a hydrazide HY in the form of a carboxylic hydrazide with an aldehyde or ketone, while the compound of the formula (XIX) is formed by the reaction of a hydrazide HY in the form of a sulfonic hydrazide with an aldehyde or ketone, the aldehyde or ketone having been released from the blocked amine BA when the composition was cured.

The cured composition AZ preferably comprises a compound of the formula (XVIII a) or (XVIII b). With particular preference the compound of the formula (XVIII a) or (XVIII b) is formed from the reaction of a carboxylic dihydrazide with an aldehyde or ketone, the carboxylic dihydrazide being preferably selected from the group consisting of carbodihydrazide, oxalic dihydrazide, succinic dihydrazide, adipic dihydrazide, suberic dihydrazide, azelaic dihydrazide, sebacic dihydrazide, dodecanoic dihydrazide and isophthalic dihydrazide. The most preferred of these is adipic dihydrazide.

Preferably Q¹ is a hydrogen atom and Q² is —C(R¹)(R²)(Z¹), where Z¹ more particularly is a radical of the formula (II) or (III) or (IV).

The present invention further provides an aldazide of the formula (XX a) or (XX b),

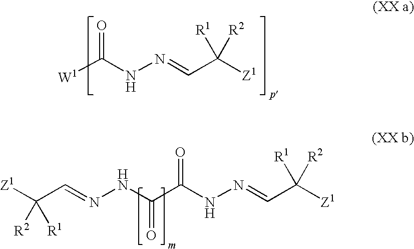

where

W¹ is the p′-valent radical of a hydrazide HY of a carboxylic acid that has a melting point of at least 100° C., more particularly of at least 150° C., following removal of p′ carboxylic hydrazide groups;

p′ is 2 or 3 or 4; and

m, R¹, R² and Z¹ have the definitions already mentioned.

Preferably p′ is 2.

Preferably Z¹ is a radical of the formula (II) or (III) or (IV).

### EXAMPLES

### Description of the Measurement Methods

Infrared spectra were measured on an FT-IR instrument 1600 from Perkin-Elmer, solid substances as pressed KBr samples in the direct beam, liquids as undiluted films, and cured polymers as pressed films on a horizontal ATR measuring unit with ZnSe crystal; the absorption bands are reported in wavenumbers (cm⁻¹) (measurement window: 4000-650 cm⁻¹); the additional note sh indicates a band which appears as a shoulder, while the additional note br indicates a broad band.

¹H-NMR spectra were measured on a Bruker DPX-300 spectrometer at 300.13 MHz; the chemical shifts δ are recorded in ppm relative to tetramethylsilane (TMS), coupling constants J are recorded in Hz. No distinction was made between true and pseudo coupling patterns.

The viscosity was measured on a Physica UM thermostated cone plate viscometer (cone diameter 20 mm, cone angle 1°, cone tip-plate distance 0.05 mm, shear rate 10 to 1000 s⁻¹).

The amine content, in other words the total amount of free amino groups and blocked amino groups (aldimino groups) in the compounds prepared, was determined by titrimetry (with 0.1N HClO₄ in glacial acetic acid, against crystal violet) and is consistently reported in mmol N/g.

Standard conditions refers to a temperature of 23±1° C. at a relative atmospheric humidity of 50±5%.

Preparation of Aldimines

Aldimine A-1

A round-bottom flask was charged under a nitrogen atmosphere with 23.0 g (0.22 mol) of benzaldehyde. With vigorous stirring, 25.0 g (0.21 mol N) of polyetherdiamine (polyoxypropylene-diamine having an average molecular weight of about 240 g/mol; Jeffamine® D-230, Huntsman; amine content 8.29 mmol N/g) were added slowly from a dropping funnel. The volatile constituents were then removed at 80° C. under reduced pressure (10 mbar, 80° C.). Yield: 44.0 g of a yellowish oil which is liquid at room temperature and has an amine content of 4.72 mmol N/g.

Aldimine A-2

A round-bottom flask was charged under a nitrogen atmosphere with 74.3 g (0.26 mol) of distilled 2,2-dimethyl-3-lauroyloxypropanal. With vigorous stirring, 30.0 g (0.25 mol N) of polyetherdiamine (polyoxypropylene-diamine having an average molecular weight of about 240 g/mol; Jeffamine® D-230, Huntsman; amine content 8.29 mmol N/g) were added slowly from a dropping funnel. The volatile constituents were then removed under reduced pressure (10 mbar, 80° C.). Yield: 99.5 g of a clear, pale yellow oil which has an amine content of 2.50 mmol N/g.

Aldimine A-3

A round-bottom flask was charged under a nitrogen atmosphere with 52.4 g (0.18 mol) of distilled 2,2-dimethyl-3-lauroyloxypropanal. With vigorous stirring, 10.0 g (0.17 mol N) of 1,6-hexamethylenediamine (BASF; amine content 17.0 mmol N/g) were added slowly from a heated dropping funnel. The volatile constituents were then removed under reduced pressure (10 mbar, 80° C.). Yield: 57.7 g of a clear, pale yellow oil which has an amine content of 2.85 mmol N/g.

### Preparation of Aldazides

### Example 1

In a round-bottom flask, under a nitrogen atmosphere, 5.00 g (0.029 mol) of finely triturated adipic dihydrazide (melting point 180-182° C.) was suspended in 17.96 g (0.063 mol) of 2,2-dimethyl-3-lauroyloxypropanal and 50 ml of absolute ethanol. The suspension was heated to 90-95° C. with vigorous stirring, in the course of which it was slightly evacuated. The volatile constituents were then removed completely by further heating to 120° C. and application of reduced pressure (5·10⁻² mbar). Yield: 21.8 g of an opalescent, colorless and odorless oil, which crystallized on standing at room temperature over a number of hours to form a white body. Melting point (uncorrected): 62-63° C.

IR: 3203 w br (ν_{N—H}), 3073 w br (ν_{N—H}), 2954 m sh, 2921 s, 2869 m sh, 2852 s, 1738 s (ν_{OC═O}), 1672 vs br (ν_{C═N}), 1630 m (ν_{NC═O}), 1553 w br (δ_{N—H}), 1485 w sh, 1465 m, 1439 m sh, 1393 m, 1376 m, 1365 m sh, 1347 w, 1298 m sh, 1282 m, 1248 m, 1233 m, 1159 s, 1112 s, 1075 w, 1059 w, 1018 m, 1002 m, 933 m, 887 w sh, 865 w, 797 w, 740 m sh, 721.

¹H-NMR (CDCl₃, 300 K): δ 9.35 (s, 2H, ═N—NH—CO), 7.05 (s, 2H, CH═N), 4.00 (s, 4H, C(CH₃)₂—CH₂—O), 2.64 (t, J≈6.8, 4H, NHC(O)—CH₂—CH₂), 2.31 (t, J≈7.5, 4H, OC(O)—CH₂—CH₂), 1.74 (t, J≈7.0, 4H, NHC(O)—CH₂—CH₂), 1.61 (m, 4H, OC(O)—CH₂—CH₂), 1.25 (m, 32H, CH₃—(CH₂)₈—CH₂—CH₂—CO), 1.14 (s, 12H, C(CH₃)₂—CH₂—O), 0.88 (t, J≈6.8, 6H, CH₃—(CH₂)₁₀—CO).

### Example 2

In a round-bottom flask, under a nitrogen atmosphere, 1.00 g (11.1 mmol) of finely triturated carbohydrazide (=carbodihydrazide; melting point 150-153° C.) was suspended in 6.63 g (23.3 mmol) of 2,2-dimethyl-3-lauroyloxypropanal. The suspension was heated to 70° C. with vigorous stirring and at the same time evacuated under a high vacuum (5·10⁻² mbar). This gave a clear, colorless oil, which solidified suddenly to form a white body. Mortar grinding and renewed evacuation under a high vacuum at 70° C. gave 7.19 g of product in the form of a white, odorless powder. Melting point (uncorrected): 85-87° C.

IR: 3190 w br (ν_{N—H}), 3086 w br (ν_{N—H}), 2948 m sh, 2914 s, 2868 m sh, 2848 s, 1730 vs (ν_{OC═O}), 1686 vs (ν_{C═N}), 1548 s (ν_{NC═O}), 1470 m, 1430 w, 1418 w, 1396 w, 1384 w, 1366 m, 1350 w, 1326 w, 1312 w, 1299 w, 1284 w, 1258 m, 1236 m, 1214 w sh, 1206 m, 1178 s, 1140 m, 1106 s, 1076 w, 1060 w, 1051 w, 1028 m, 1000 m, 972 w, 930 m, 875 w, 831 vw, 810 w, 790 w, 775 vw, 746 m, 728 m sh, 718 m, 664 w.

From examples 1 and 2 it is evident that the aldehyde 2,2-dimethyl-3-lauroyloxypropanal reacts in each case with a carboxylic hydrazide having a melting point of at least 100° C., at elevated temperature and with high conversion to form the corresponding aldazide.

Hydrazides Used

| | | |
|---|---|---|
| Hydrazide HY-1: | adipic dihydrazide | melting point 180-182° C. |
| Hydrazide HY-2: | benzohydrazide | melting point 112-114° C. |
| Hydrazide HY-3: | 2-thiophene- | melting point 136-139° C. |
| | carbohydrazide | |

### Preparation of Polyurethane Compositions

### Examples 3 to 7 and Comparative Examples 8 to 9

In a polypropylene beaker with screw closure, the polymer P-1, whose preparation is described below, was mixed to a homogeneous material by means of a centrifugal mixer (SpeedMixer™ DAC 150, FlackTek Inc.; 1 min at 2500 rpm) with the added ingredients listed in table 1, in the parts by weight indicated.

The polymer P-1 was prepared as follows:

1300 g of polyoxypropylene diol (Acclaim® 4200 N, Bayer; OH Number 28.5 mg KOH/g), 2600 g of polyoxypropylene-polyoxyethylene triol (Caradol® MD34-02, Shell; OH number 35.0 mg KOH/g), 600 g of 4,4′-methylenediphenyl diisocyanate (4,4′-MDI; Desmodur® 44 MC L, Bayer) and 500 g of diisodecyl phthalate (DIDP; Palatinol® Z, BASF) were reacted at 80° C. to form an NCO-terminated polyurethane polymer having a free isocyanate group content of 2.05% by weight.

TABLE 1
Composition of examples 3 to 7 and of comparative examples 8 to 9.

| | Example | | | | | | |
|---|---|---|---|---|---|---|---|
| | 3 | 4 | 5 | 6 | 7 | 8 (comp.) | 9 (comp.) |
| Polymer P-1 | 50.0 | 50.0 | 50.0 | 50.0 | 50.0 | 50.0 | 50.0 |
| Aldimine A-1 | — | — | — | — | 2.58 | — | — |
| Aldimine A-2 | 4.89 | 4.89 | 4.89 | 4.89 | — | 4.89 | — |
| Hydrazide HY-1 | 1.06 | 0.53 | — | — | 1.06 | — | — |
| Hydrazide HY-2 | — | — | 1.67 | — | — | — | — |
| Hydrazide HY-3 | — | — | — | 1.75 | — | — | — |
| Acid catalyst^{a} | 0.50 | 0.50 | 0.50 | 0.50 | 0.50 | 0.50 | 0.50 |
| Amine catalyst^{b} | 0.05 | 0.05 | 0.05 | 0.05 | 0.05 | 0.05 | 0.05 |
| [Aldimine/NCO]^{c} | 0.50 | 0.50 | 0.50 | 0.50 | 0.50 | 0.50 | 0 |
| [Hydrazide/ | 1 | 0.5 | 1 | 1 | 1 | 0 | — |
| aldimine]^{d} | | | | | | | |

^{a}5% by weight salicylic acid in dioctyl adipate.
^{b}2,2′-dimorpholinodiethyl ether (DABCO ® DMDEE catalyst, Air Products).
^{c}Ratio between aldimino groups and isocyanate groups.
^{d}Ratio between hydrazide groups and aldimino groups.

The compositions thus obtained were tested for viscosity, storage stability, skinning time, blistering, mechanical properties, outgassing loss, and degree of outgassing of the aldehyde.

The storage stability was measured via the change (increase) in viscosity during storage under hot conditions. For this purpose, the composition was stored in a sealed tube in an oven at 60° C., and the viscosity at 20° C. was measured a first time after 6 hours, and a second time after 7 days of storage. The storage stability is indicated by the percentage increase in the second viscosity value relative to the first.

For the measurement of the skinning time (time to absence of tack, tack-free time) a few grams of the composition, after having been stored at 60° C. for six hours and now at room temperature, were applied in a layer thickness of about 2 mm to cardboard, and, under standard conditions, a measurement was made of the time which elapsed until for the first time there were no residues of the composition remaining on an LDPE pipette contacted gently with the surface of the composition.

The blistering was assessed qualitatively on the basis of the amount of blisters which occurred in the course of the curing of the composition.

Mechanical properties measured were the tensile strength (breaking force), the elongation at break and the elasticity modulus of the cured composition. For this purpose, the composition, stored at 60° C. for 6 hours and now at room temperature, was cast in a flat PTFE mold to form a film with a thickness of about 2 mm, which was cured under standard conditions for 7 days. Dumbbell specimens with a length of 75 mm, the narrow central part having a length of 30 mm and a width of 4 mm, were punched from the film, and were tested in accordance with DIN EN 53504 with a pulling speed of 200 m/min.

The outgassing loss of the cured composition was determined by punching dumbbell specimens of the same dimensions from the film produced as described above, storing them open in an oven at 100° C. for seven days, and then testing them for weight loss relative to their initial weight (report in % by weight). The value reported is in each case the average of three dumbbell specimens of the same film.

The degree of outgassing of the aldehyde was calculated from the outgassing loss of the cured composition, corrected to take account of the outgassing loss of the aldimine-free comparative example 9 (“blank value”), in accordance with the following formula: degree of outgassing of the aldehyde=[(outgassing loss−outgassing loss ex. 9)/aldehyde content]×100%. The aldehyde content of a composition can be calculated directly from the aldimine content. The lower the degree of outgassing of the aldehyde, the less aldehyde outgassed.

For the determination of the fogging behavior, the procedure was as follows: a section of the film produced as described above was cut into sections with a size of about 2×2 mm. Approximately 5 g of these sections were weighed into a crystallizing boat, which was covered with a tarred watch glass, and the boat was heated, with a low depth of immersion, in an oil bath at 100° C. for 24 hours or at 130° C. for 12 hours, a coating of condensation forming on the watch glass. The amount of condensation was determined by re-weighing the coated watch glass, and was reported in percent of the amount of film weighed out as “fogging”.

The propensity toward yellowing was evaluated qualitatively on a scale of 0 to 3, relative to an unheated reference sample, on samples stored at 100° C. for 7 days, with “0” denoting no yellowing, “1” denoting slight yellowing (pale yellow), “2” denoting moderate yellowing (deep yellow) and “3” denoting severe yellowing (brown-yellow).

The results of the tests are listed in table 2.

TABLE 2
Properties of examples 3-7 and of comparative examples 8-9.

| | Example | | | | | | |
|---|---|---|---|---|---|---|---|
| | 3 | 4 | 5 | 6 | 7 | 8 (comp.) | 9 (comp.) |
| Viscosity after 6 h^{a} | n.d. | n.d. | 32 | 27 | n.d. | n.d. | n.d. |
| | 35 | 33 | n.d. | n.d. | 49 | 32 | 60 |
| Viscosity after 7 d^{a} | n.d. | n.d. | 55 | 42 | n.d. | n.d. | n.d. |
| | 40 | 38 | n.d. | n.d. | 83 | 37 | 63 |
| Viscosity increase (%)^{b} | 14 | 15 | 72 | 56 | 69 | 16 | 5 |
| Skinning time (min) | 18 | 20 | 21 | 19 | 55 | 24 | 45 |
| Blistering^{c} | ok | ok | ok | ok | B | ok | BB |
| Tensile strength (MPa)^{d} | 0.54 | 0.55 | 0.44 | 0.67 | 0.42 | 0.50 | 0.69 |
| | 0.72 | 0.93 | 0.63 | 0.90 | 0.65 | 0.71 | 0.71 |
| Elongation at break (%)^{d} | 188 | 191 | 240 | 214 | 349 | 133 | 86 |
| | 321 | 345 | 319 | 277 | 667 | 186 | 93 |
| Elasticity modulus at | 0.59 | 0.50 | 0.26 | 0.34 | 0.10 | 0.50 | 1.08 |
| 0.5-5% elongation (MPa)^{d} | 0.42 | 0.52 | 0.38 | 0.60 | 0.25 | 0.64 | 0.95 |
| Outgassing loss after 7 d/ | 3.28 | 5.40 | 3.13 | 6.00 | 2.13 | 7.95 | 1.33 |
| 100° C. (%) | | | | | | | |
| Degree of outgassing of | 30 | 62 | 28 | 73 | 33 | 100 | — |
| the aldehyde (%) | | | | | | | |
| Fogging after | 0.02 | n.d. | n.d. | n.d. | n.d. | 0.55 | 0.01 |
| 24 h/100° C. (%) | | | | | | | |
| Fogging after | 0.12 | n.d. | n.d. | n.d. | n.d. | 2.30 | 0.05 |
| 12 h/130° C. (%) | | | | | | | |
| Yellowing after 7 d/100° C. | 1 | 1-2 | 1-2 | 1-2 | 1-2 | 3 | 2-3 |

^{a}in Pa · s, 1st number: storage at room temperature, 2nd number: storage at 60° C.
^{b}= [(Viscosity after 7 d/viscosity after 6 h) − 1] × 100%.
^{c}ok = no blisters; B = blisters; BB = lots of blisters.
^{d}Upper number: testing after curing under standard conditions; lower number: testing after heat loading of the cured material (7 d/100° C.).
n.d. = not determined.

From table 2 it is evident that the inventive compositions of examples 3 to 7, which comprise a hydrazide as well as an aldimine, relative to the non-hydrazide composition of comparative example 8, have a slightly to significantly reduced outgassing loss, a lower fogging, and a low propensity toward yellowing. Depending on the hydrazide used, differences are apparent in the storage stability; examples 3, 4 and 7 are storage-stable at 60° C., while examples 5 and 6 are storage-stable at room temperature. Reference example 9, which contains no aldimine, shows a low outgassing loss and low fogging, but has a propensity toward blistering and yellowing.

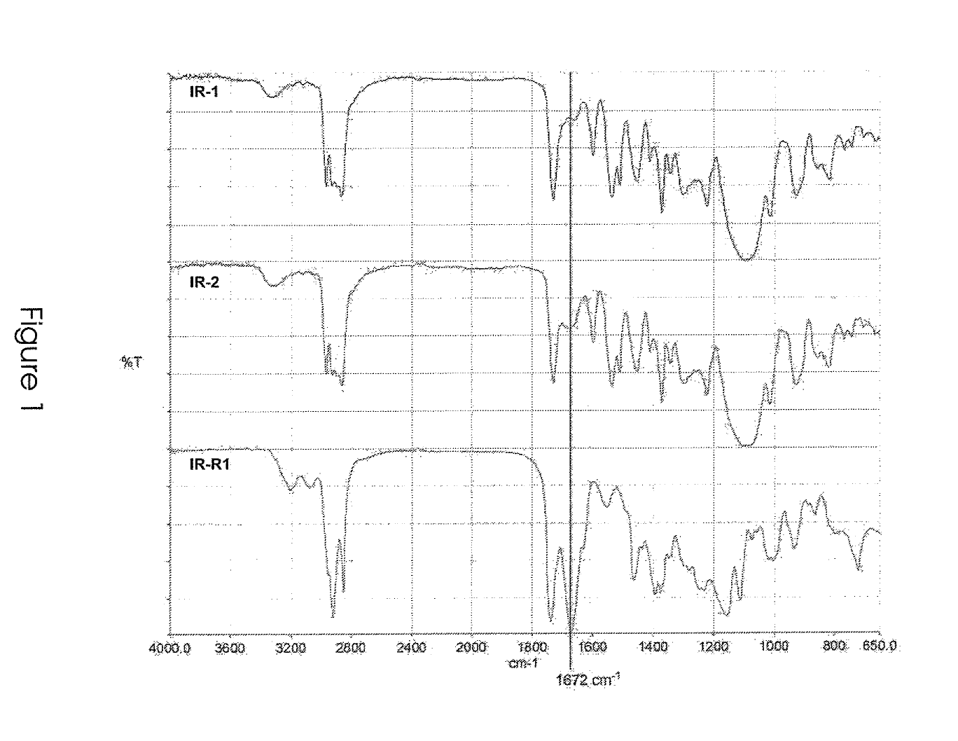

FIG. 1 shows infrared spectra. The spectrum labeled as IR-1 was recorded from the polyurethane composition of example 3 after curing under standard conditions. The spectrum labeled as IR-2 was recorded from the polyurethane composition of example 3 after curing under standard conditions and subsequent heating at 100° C. for 24 hours. The spectrum labeled as IR-R1 is the spectrum of the aldazide of example 1 (condensation product of adipic dihydrazide and 2,2-dimethyl-3-lauroyloxypropanal).

From a comparison of these three spectra, more particularly through the band at 1672 cm⁻¹ (ν_{C═N}) which is characteristic for the aldazide, it is evident that the heating of the polyurethane composition of example 3 has formed the aldazide of example 1. The heating of the film cured under standard conditions to 100° C. has therefore resulted in a significant conversion of the adipic dihydrazide with the aldehyde released from the aldimine A-2 (2,2-dimethyl-3-lauroyloxypropanal) to form the corresponding aldazide.

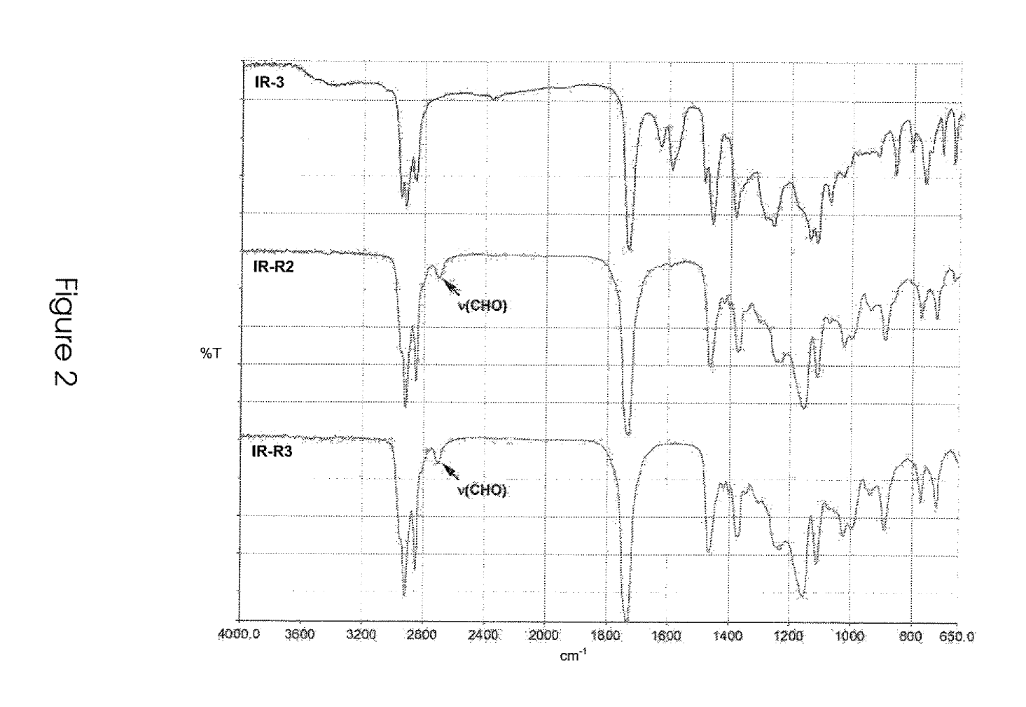

For examples 3 and 8, an infrared spectrum was recorded after the determination of the fogging at 130° C. from the respective condensation on the watch glass. In FIG. 2, the spectrum labeled as IR-3 shows the condensate from example 3, while the spectrum labeled as IR-R2 shows the condensate from example 8. The spectrum labeled as IR-R3 originates from 2,2-dimethyl-3-lauroyloxypropanal, i.e. from the aldehyde released from the aldimine A-2. From a comparison of these spectra it is evident that the condensate of example 8 represents substantially pure 2,2-dimethyl-3-lauroyloxypropanal, whereas the quantitatively significantly lower (cf. table 2) condensate of example 3, in view of the absence of the marked aldehyde-typical band (ν_{CHO}), obviously contains little or no aldehyde.

### Preparation of One-Pack Polyurethane Adhesives

### Examples 10 to 12 and Comparative Examples 13 to 14

### Elastic Glazing Adhesive

For each example, the respective ingredients as per table 3 were processed in the indicated parts by weight, in a vacuum mixer and in the absence of moisture, to form a homogeneous paste, which was immediately dispensed into an internally coated aluminum cartridge and the cartridge was given an airtight seal. The polymer P-1 was prepared as described in example 3.

TABLE 3
Composition of the adhesives of examples 10 to 12 and of
comparative examples 13 to 14.

| | Example | | | | |
|---|---|---|---|---|---|
| | 10 | 11 | 12 | 13 (comp.) | 14 (comp.) |
| Polymer P-1 | 40.00 | 40.00 | 40.00 | 40.00 | 40.00 |
| Aldimine A-2 | 5.47 | 5.47 | — | 5.47 | — |
| Aldimine A-3 | — | — | 4.79 | — | — |
| Hydrazide HY-1 | 1.19 | 0.59 | 1.19 | — | — |
| Plasticizer^{a} | 15.53 | 15.53 | 16.21 | 15.53 | 21.00 |
| Kaolin^{b} | 20.81 | 21.41 | 20.81 | 22.00 | 22.00 |
| Carbon black^{b} | 12.00 | 12.00 | 12.00 | 12.00 | 12.00 |
| Thickener^{c} | 4.00 | 4.00 | 4.00 | 4.00 | 4.00 |
| Drying agent^{d} | 0.20 | 0.20 | 0.20 | 0.20 | 0.20 |
| Epoxysilane^{e} | 0.20 | 0.20 | 0.20 | 0.20 | 0.20 |
| Acid catalyst^{f} | 0.50 | 0.50 | 0.50 | 0.50 | 0.50 |
| Amine catalyst^{g} | 0.10 | 0.10 | 0.10 | 0.10 | 0.10 |
| [Aldimine/NCO]^{h} | 0.70 | 0.70 | 0.70 | 0.70 | 0 |
| [Hydrazide/aldimine]ⁱ | 1 | 0.5 | 1 | 0 | — |

^{a}Diisodecyl phthalate (DIDP; Palatinol ® Z, BASF).
^{b}Dried at 130° C.
^{c}Hydrophobic fumed silica (Aerosil ® R972, Degussa).
^{d}p-Toluenesulfonyl isocyanate (additive TI, Bayer).
^{e}3-Glycidyloxypropyltriethoxysilane (Dynasylan ® GLYEO, Degussa).
^{f}5% by weight salicylic acid in dioctyl adipate.
^{g}2,2′-Gimorpholinodiethyl ether (DABCO ® DMDEE catalyst, Air Products).
^{h}Ratio between aldimino groups and isocyanate groups.
ⁱRatio between hydrazide groups and aldimino groups.

The adhesives thus obtained were tested for application properties, skinning time, mechanical properties, outgassing loss and degree of outgassing of the aldehyde.

As a measure of the application properties the sag resistance and the stringing were employed. For the determination of the sag resistance, the adhesive was applied using a cartridge gun via a triangular nozzle, in the form of a horizontally extending triangular bead having a base diameter of 8 mm and a height (distance of the peak of the triangle from the base) of 20 mm, onto a vertical piece of cardboard. After 5 minutes, a measurement was made of the extent to which the peak had dropped, i.e. had moved down from the original position in the middle of the triangular bead. The evaluation was “very good” if the peak was in an entirely or approximately unchanged position, and “good” when the peak was situated between the middle and the end of the base. The stringing was determined qualitatively, by applying an amount of adhesive, using a cartridge gun, to a piece of cardboard affixed to the wall, moving the cartridge gun away from the applied adhesive at the end of application, in a rapid withdrawal movement, and measuring the length of the string remaining at the tear point.

For the determination of the mechanical properties after curing measurements were made of the Shore A hardness, tensile strength, elongation at break and elasticity modulus. The Shore A hardness was determined in accordance with DIN 53505 on test specimens cured under standard conditions for 14 days.

The remaining properties were tested as described, for example 3, with the outgassing loss also determined after 7 d/180° C. For the calculation of the degree of outgassing, the blank value used was the outgassing loss of the aldimine-free comparative example 14.

All of the adhesives cured completely without blisters.

The results of the tests are set out in table 4.

TABLE 4
Properties of the adhesives of examples 10 to 12 and of
comparative examples 13 to 14.

| | Example | | | | |
|---|---|---|---|---|---|
| | | | | 13 | 14 |
| | 10 | 11 | 121 | (comp.) | (comp.) |
| Sag resistance | Very | Very | Good | Very | Very |
| | good | good | | good | good |
| Stringing (cm) | 0.3 | 0.3 | 1.2 | 1.0 | 1.0 |
| Skinning time (min) | 26 | 23 | 24 | 26 | 115 |
| Shore A hardness | 51 | 52 | 62 | 49 | 59 |
| Tensile strength (MPa)^{a} | 6.4 | 6.3 | 5.0 | 6.5 | 6.6 |
| | 5.8 | 6.4 | 6.5 | 6.8 | 7.5 |
| | 6.0 | 6.7 | 5.0 | 6.7 | 7.0 |
| | 5.9 | 6.6 | 4.8 | 7.3 | 7.5 |
| Elongation at break (%)^{a} | 650 | 660 | 530 | 650 | 470 |
| | 520 | 510 | 300 | 470 | 370 |
| | 670 | 620 | 420 | 570 | 400 |
| | 580 | 550 | 290 | 450 | 320 |
| Elasticity modulus at | 2.8 | 2.9 | 3.9 | 2.7 | 3.6 |
| 0.5-5% elongation | 2.1 | 2.3 | 4.2 | 1.8 | 3.4 |
| (MPa)^{a} | 2.3 | 3.1 | 4.2 | 3.5 | 4.0 |
| | 2.2 | 2.9 | 3.8 | 4.2 | 4.2 |
| Outgassing loss (%)^{b} | 1.55 | 2.53 | 1.47 | 4.03 | 0.88 |
| | 1.84 | 3.07 | 1.80 | 5.36 | 1.80 |
| Degree of outgassing of | 16 | 40 | 14 | 77 | — |
| the aldehyde (%)^{b} | 1 | 31 | 0 | 87 | — |

^{a}1st number: testing after curing under standard conditions; 2nd number: testing after condensation exposure (7 d/70° C.) (“hot/wet”) of the material cured under standard conditions; 3rd number: testing after heat loading (7 d/80° C.) of the material cured under standard conditions; 4th number: testing after heat loading (7 d/100° C.) of the material cured under standard conditions.
^{b}Top number: after 7 d/80° C.; bottom number: after 7 d/100° C.

From Table 4 it is evident that the inventive adhesives of examples 10 to 12, which comprise a hydrazide as well as an aldimine, have a sharply reduced outgassing loss as compared with the non-hydrazide adhesive of comparative example 13. When the hydrazide is used stoichiometrically in relation to the aldimine (examples 10 and 11), outgassing losses at 100° C. are obtained which are close to those of the reference example 14 without aldimine; the calculated degree of outgassing of the aldehyde in this case sinks to close to 0%, which means that there is virtually no longer any aldehyde escaping.

### Examples 15 to 16 and Comparative Examples 17 to 18

### Elastic Assembly Adhesive

For each example, the respective ingredients as per table 5, in the parts by weight indicated, were processed in a vacuum mixer and in the absence of moisture to form a homogeneous paste, which is immediately dispensed into an internally coated aluminum cartridge, which was given an airtight seal.

The polymer P-1 was prepared as described in example 3.

The polyurethane polymer P-2 was prepared as follows:

590 g of Acclaim® 4200 N polyol (polypropylene oxide diol, OH number 28.5 mg KOH/g; Bayer), 1180 g of Caradol® MD34-02 polyol (polypropylene oxide-polyethylene oxide triol, OH number 35.0 mg KOH/g; Shell) and 230 g of isophorone diisocyanate (IPDI; Vestanat® IPDI, Degussa) were reacted by a known method at 80° C. to give an NCO-terminated polyurethane polymer having a titrimetrically determined free isocyanate group content of 2.1% by weight.

The urea thickener was prepared as follows:

A vacuum mixer was charged with 3000 g of diisodecyl phthalate (DIDP; Palatinol® Z, BASF) and 480 g of 4,4′-methylenediphenyl diisocyanate (4,4′-MDI; Desmodur® 44 MC L, Bayer), and this initial charge was gently heated. Then, with vigorous stirring, 270 g of monobutylamine were added slowly dropwise. The resultant paste was stirred for a further hour under reduced pressure and with cooling.

TABLE 5
Composition of the adhesives of examples 15 to 16 and of
comparative examples 17 to 18.

| | Example | | | |
|---|---|---|---|---|
| | | | 17 | 18 |
| | 15 | 16 | (Comp.) | (Comp.) |
| Polymer P-1 | 30.00 | 30.00 | 30.00 | 30.00 |
| Polymer P-2 | 5.00 | 5.00 | 5.00 | 5.00 |
| Aldimine A-2 | 4.81 | 4.81 | 4.81 | — |
| Hydrazide HY-1 | 1.04 | 0.52 | — | — |
| Chalk | 29.96 | 30.48 | 31.00 | 31.00 |
| Thickener^{a} | 23.74 | 23.74 | 23.74 | 28.55 |
| Titanium dioxide | 4.50 | 4.50 | 4.50 | 4.50 |
| Epoxysilane^{b} | 0.25 | 0.25 | 0.25 | 0.25 |
| Acid catalyst^{c} | 0.50 | 0.50 | 0.50 | 0.50 |
| Tin catalyst^{d} | 0.10 | 0.10 | 0.10 | 0.10 |
| Amine catalyst^{e} | 0.10 | 0.10 | 0.10 | 0.10 |
| [Aldimine/NCO]^{f} | 0.70 | 0.70 | 0.70 | 0 |
| [Hydrazide/aldimine]^{g} | 1 | 0.5 | 0 | — |

^{a}Urea thickener.
^{b}3-Glycidyloxypropyltriethoxysilane (Dynasylan ® GLYEO, Degussa).
^{c}5% by weight salicylic acid in dioctyl adipate.
^{d}Dibutyltin dilaurate (5% by weight in diisodecyl phthalate).
^{e}2,2′-Dimorpholinodiethyl ether (DABCO ® DMDEE catalyst, Air Products).
^{f}Ratio between aldimino groups and isocyanate groups.
^{g}Ratio between hydrazide groups and aldimino groups.

The adhesives thus obtained were tested for application properties, skinning time, mechanical properties, outgassing loss and degree of outgassing of the aldehyde, as described for example 10. For the calculation of the degree of outgassing, the blank value used was the outgassing loss of the aldimine-free comparative example 18. Moreover, the blistering was tested as described for example 3.

The results of the tests are set out in table 6.

TABLE 6
Properties of the adhesives of examples 15 to 16 and the
comparative examples 17 to 18.

| Example | | | |
|---|---|---|---|
| | | 17 | 18 |
| 15 | 16 | (Comp.) | (Comp.) |

| | | | | |
|---|---|---|---|---|
| Sag resistance | very | very | very | very |
| | good | good | good | good |
| Stringing (cm) | 10 | 11 | 8 | 9 |
| Skinning time (min) | 30 | 28 | 37 | 46 |
| Blistering | none | none | none | some |
| Shore A hardness | 40 | 42 | 41 | 50 |
| Tensile strength (MPa)^{a} | 1.45 | 1.47 | 1.80 | 1.70 |
| | 1.91 | 1.60 | 1.83 | n.d. |
| | 1.91 | 1.86 | 2.11 | n.d. |
| | 1.88 | 2.10 | 2.23 | n.d. |
| Elongation at break (%)^{a} | 720 | 730 | 840 | 275 |
| | 730 | 750 | 790 | n.d. |
| | 920 | 910 | 890 | n.d. |
| | 1080 | 1080 | 1030 | n.d. |
| Elasticity modulus at | 2.35 | 2.40 | 2.27 | 8.35 |
| 0.5-5% elongation | 2.55 | 2.51 | 1.92 | n.d. |
| (MPa)^{a} | 2.28 | 1.93 | 3.26 | n.d. |
| | 2.62 | 2.79 | 3.63 | n.d. |
| Outgassing loss (%)^{b} | 1.36 | 2.02 | 2.91 | 0.74 |
| | 1.72 | 3.02 | 4.08 | 1.45 |
| Degree of outgassing of | 17 | 36 | 61 | — |
| the aldehyde (%)^{b} | 8 | 44 | 73 | — |

^{a}1st number: testing after curing under standard conditions; 2nd number: testing after condensation exposure (7 d/70° C.) (“hot/wet”) of the material cured under standard conditions; 3rd number: testing after heat loading (7 d/80° C.) of the material cured under standard conditions; 4th number: testing after heat loading (7 d/100° C.) of the material cured under standard conditions.
^{b}Top number: after 7 d/80° C.; bottom number: after 7 d/100° C.
n.d. = not determined.

From table 6 it is evident that the inventive adhesives of examples 15 to 16, which as well as the aldimine A-2 contain adipic dihydrazide, have a significantly reduced outgassing loss as compared with the non-hydrazide adhesive of comparative example 17. When hydrazide is used stoichiometrically relative to the aldimine (example 15), the outgassing loss at 80° C. sinks to below half of the value measured for comparative example 17, and at 100° C. comes close to the value measured for reference example 18, which contains no aldimine. The thermal stability of the adhesives of examples 15 and 16 is at the same time good, comparably to that of comparative example 17.

1. A curable composition comprising
a) at least one polyisocyanate P,
b) at least one aldehyde or ketone-blocked amine BA,
c) at least one hydrazide HY of a carboxylic acid or sulfonic acid, which has a melting point of at least 100° C.,
with the proviso that the hydrazide HY is present in an amount of 0.3 to 1.1 equivalents of hydrazide groups per equivalent of aldehyde groups or keto groups, with which the amine BA is blocked.

2. The curable composition of claim 1, wherein the polyisocyanate P is a polyurethane polymer PUP which contains isocyanate groups.

3. The curable composition of claim 1, wherein the polyisocyanate P is a polyisocyanate PI in the form of a monomeric di- or triisocyanate or of an oligomer of a monomeric diisocyanate.

4. The curable composition of claim 1, wherein the polyisocyanate P is a polyisocyanate PI in the form of a form of MDI which is liquid at room temperature, or of a form of polymeric MDI (PMDI).

5. The curable composition of claim 1, wherein the blocked amine BA is an aldimine BA1 of the formula (I), 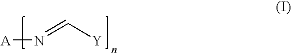
where A is the radical of an amine B following removal of n primary amino groups, Y is an organic radical having 1 to 35 C atoms and optionally containing heteroatoms, and
n is an integer from 1 to 5.

6. The curable composition of claim 5, wherein the blocked amine BA is an aldimine BA1 of the formula (I a) or (I b), 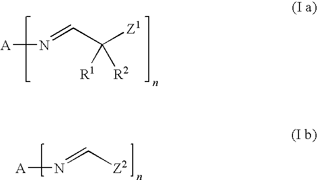
where
R¹ and R² either
independently of one another are each a monovalent hydrocarbon radical having 1 to 12 C atoms,
or
together are a divalent hydrocarbon radical having 4 to 20 C atoms which is part of an optionally substituted carbocyclic ring having 5 to 8 atoms;
Z¹ is a monovalent hydrocarbon radical having 1 to 32 C atoms which optionally contains at least one heteroatom;
Z² either
is a substituted or unsubstituted aryl or heteroaryl group which has a ring size of 5 to 8 atoms,
or is 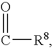
where R⁸ is a hydrogen atom or is an alkoxy group, or is a substituted or unsubstituted alkenyl or arylalkenyl group having at least 6 C atoms.

7. The curable composition of claim 6, wherein R¹ and R² in formula (I a) are each a methyl group.

8. The curable composition of claim 6, wherein Z¹ in formula (I a) is a radical of the formula (II) or (III) or (IV), 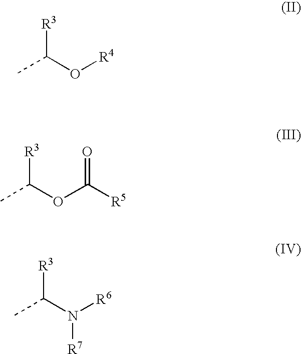
where
R³ is a hydrogen atom or is an alkyl group or is a cycloalkyl group or is an arylalkyl group having 1 to 12 C atoms;
R⁴ is a hydrocarbon radical having 1 to 30 C atoms, which optionally contains ether oxygen atoms;
R⁵ alternatively
is a hydrogen atom,
or
is a linear or branched alkyl radical having 1 to 30 C atoms, optionally with cyclic fractions and optionally with at least one heteroatom,
or
is a singly or multiply unsaturated, linear or branched hydrocarbon radical having 5 to 30 C atoms,
or
is an optionally substituted aromatic or heteroaromatic 5- or 6-membered ring;
R⁶ and R⁷ either
independently of one another are each a monovalent aliphatic, cycloaliphatic or arylaliphatic radical having 1 to 20 C atoms and optionally containing heteroatoms in the form of ether oxygen or tertiary amine nitrogen,
or
together are a divalent aliphatic radical having 3 to 20 C atoms, which is part of an optionally substituted heterocyclic ring having 5 to 8 ring atoms and containing optionally, in addition to the nitrogen atom, further heteroatoms in the form of ether oxygen or tertiary amine nitrogen.

9. The curable composition of claim 8, wherein R⁴ is a hydrocarbon radical having 6 to 30 C atoms, which optionally contains ether oxygen atoms.

10. The curable composition of claim 8, wherein R⁵ is a linear or branched alkyl radical having 6 to 30 C atoms, optionally with cyclic fractions and optionally with at least one heteroatom, or is a singly or multiply unsaturated, linear or branched hydrocarbon radical having 6 to 30 C atoms.

11. The curable composition of claim 5, wherein the amine B has at least two primary amino groups and is selected from the group consisting of 1,6-hexamethylenediamine, 1,5-diamino-2-methylpentane (MPMD), 1,3-pentanediamine (DAMP), 1-amino-3-aminomethyl-3,5,5-trimethylcyclohexane (=isophoronediamine or IPDA), 2,2,4- and 2,4,4-trimethylhexamethylenediamine (TMD), 1,3-xylylenediamine, 1,3-bis(aminomethyl)cyclohexane, bis(4-aminocyclohexyl)methane, bis(4-amino-3-methylcyclohexyl)methane, 3(4),8(9)-bis(aminomethyl)tricyclo[5.2.1.0^{2,6}]decane, 1,2-, 1,3- and 1,4-diaminocyclohexane, 1,4-diamino-2,2,6-trimethylcyclohexane, 3,6-dioxaoctane-1,8-diamine, 4,7-dioxadecane-1,10-diamine, 4-aminomethyl-1,8-octanediamine, polyoxyalkylene-polyamines having two or three amino groups, 1,3- and 1,4-phenylenediamine, 2,4- and 2,6-tolylenediamine, 4,4′-, 2,4′- and 2,2′-diaminodiphenylmethane, 3,3′-dichloro-4,4′-diaminodiphenylmethane, and mixtures of the stated polyamines.

12. The curable composition of claim 5, wherein the amine B has at least one primary amino group and at least one further reactive group, which represents alternatively a hydroxyl group, a secondary amino group or a mercapto group.

13. The curable composition of claim 1, wherein the blocked amine BA is a ketimine BA2 of the formula (VII), 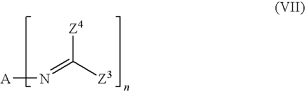
where A is the radical of an amine B following removal of n primary amino groups,
Z³ and Z⁴ either
independently of one another are each a monovalent hydrocarbon radical having 1 to 12 C atoms,
or
together are a divalent hydrocarbon radical having 4 to 20 C atoms which is part of an optionally substituted carbocyclic ring having 5 to 8 C atoms; and n is an integer from 1 to 5.

14. The curable composition of claim 1, wherein the blocked amine BA is an enamine BA3, which has at least one enamino group of the formula (IX), 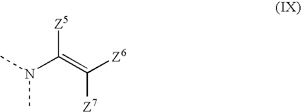
where
Z⁵ and Z⁶ either
independently of one another are each a hydrogen atom or a monovalent hydrocarbon radical having 1 to 12 C atoms,
or together are a divalent hydrocarbon radical having 3 to 20 C atoms, which is part of an optionally substituted carbocyclic ring having 5 to 8 C atoms; and
Z⁷ is a hydrogen atom or a monovalent hydrocarbon radical having 1 to 12 C atoms.

15. The curable composition of claim 1, wherein the blocked amine BA is an oxazolidine BA4 of the formula (XI), 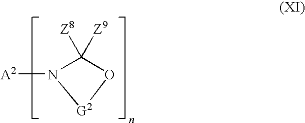
where
A² is the radical of an amine following removal of n secondary amino groups;
G² is an optionally substituted C₂ or C₃ alkylene radical;
Z⁸ and Z⁹ independently of one another are each a hydrogen atom or a monovalent hydrocarbon radical having 1 to 12 C atoms;
and n is an integer from 1 to 5.

16. The curable composition of claim 1, wherein the hydrazide HY has the formula (XIV a) or (XIV b) or (XV), 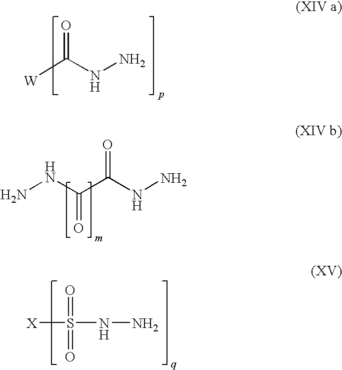
where
W is the p-valent radical of a hydrazide HY of a carboxylic acid, which has a melting point of at least 100° C. following removal of p carboxylic hydrazide groups;
X is the q-valent radical of a hydrazide HY of a sulfonic acid, which has a melting point of at least 100° C. following removal of q sulfonic hydrazide groups;
m is zero or 1;
p is 1 or 2 or 3 or 4; and
q is 1 or 2 or 3 or 4.

17. The curable composition of claim 1, wherein the hydrazide HY is a carboxylic hydrazide.

18. The curable composition of claim 1, wherein the hydrazide HY is present in an amount of 0.3 to 1.1 equivalent(s) of hydrazide groups per equivalent of aldehyde or keto groups, with which the amine BA is blocked.

19. A method of adhesively bonding a substrate S1 to a substrate S2, which comprises the steps of:
i) applying a curable composition of claim 1 to a substrate S1 at a temperature below 40° C.;
ii) contacting the applied composition with a substrate S2 within the open time of the composition;
iii) curing the applied composition at a temperature below 40° C.;
or
i′) applying the curable composition to a substrate S1 and to a substrate S2 at a temperature below 40° C.;
ii′) contacting the applied composition with one another within the open time of the composition;
iii′) curing the applied composition at a temperature below 40° C.; the substrate S2 being composed of the same material as or a different material from the substrate S1.

20. A method of sealing comprising the steps of:
i″) applying a curable composition of claim 1 at a temperature below 40° C. between a substrate S1 and a substrate S2, so that the composition is in contact with the substrate S1 and the substrate S2;
ii″) curing the applied composition at a temperature below 40° C.; the substrate S2 being composed of the same material as or a different material from the substrate S1.

21. A method of coating a substrate S1 comprising the steps of:
i′″) applying a curable composition of claim 1 at a temperature below 40° C. to a substrate S1 within the open time of the composition;
ii′″) curing the applied composition at a temperature below 40° C.

22. An article obtained by a method of claim 19.

23. A cured composition obtained by curing a curable composition of claim 1 at a temperature below 40° C. by means of exposure to water, and subsequently heating the thus cured composition to a temperature of 80° C. or higher;
and this cured composition comprising at least one compound of the formula (XVIII a) or (XVIII b) or (XIX), 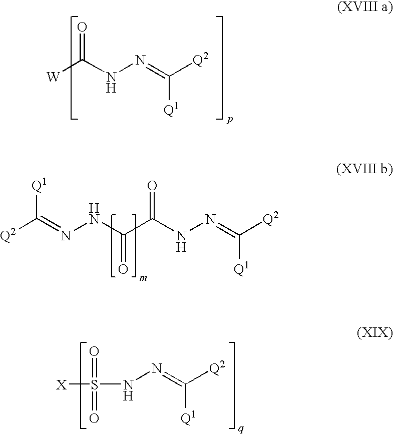
where
W is the p-valent radical of a hydrazide HY of a carboxylic acid, which has a melting point of at least 100° C. following removal of p hydrazide groups;
X is the q-valent radical of a hydrazide HY of a sulfonic acid, which has a melting point of at least 100° C. following removal of q sulfonic hydrazide groups;
m is zero or 1;
p is 1 or 2 or 3 or 4; and
q is 1 or 2 or 3 or 4;
and
Q¹ is a hydrogen atom or is Z³ or Z⁵ or Z⁸,
where
if Q¹ is a hydrogen atom, Q² is Y,
or
if Q¹ is Z³, Q² is Z⁴,
or
if Q¹ is Z⁵, Q² is 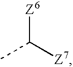
or
if Q¹ is Z⁸, Q² is Z⁹,
where
Y is an organic radical having 1 to 35 C atoms, which optionally contains heteroatoms;
Z³ and Z⁴ either
independently of one another are each a monovalent hydrocarbon radical having 1 to 12 C atoms,
or
together are a monovalent hydrocarbon radical having 4 to 20 C atoms, which is part of an optionally substituted carbocyclic ring having 5 to 8 C atoms;
Z⁵ and Z⁶ either
independently of one another are each a hydrogen atom or a monovalent hydrocarbon radical having 1 to 12 C atoms,
or together are a divalent hydrocarbon radical having 3 to 20 C atoms which is part of an optionally substituted carbocyclic ring having 5 to 8 C atoms;
Z⁷ is a hydrogen atom or is a monovalent hydrocarbon radical having 1 to 12 C atoms; and
Z⁸ and Z⁹ independently of one another are each a hydrogen atom or a monovalent hydrocarbon radical having 1 to 12 C atoms.

24. An aldazide of the formula (XX a) or (XX b), 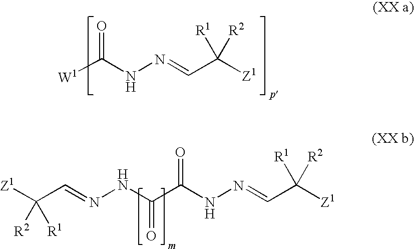
where
W¹ is the p′-valent radical of a hydrazide HY of a carboxylic acid, which has a melting point of at least 100° C. following removal of p′ hydrazide groups;
R¹ and R² either
independently of one another are each a monovalent hydrocarbon radical having 1 to 12 C atoms,
or
together are a divalent hydrocarbon radical having 4 to 20 C atoms, which is part of an optionally substituted carbocyclic ring having 5 to 8 C atoms;
Z¹ is a monovalent hydrocarbon radical having 1 to 32 C atoms, which optionally contains at least one heteroatom;
p′ is 2 or 3 or 4; and
m is zero or 1.

25. The aldazide of claim 24, wherein Z¹ is a radical of the formula (II), 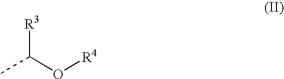
where R³ is a hydrogen atom or is an alkyl group or is a cycloalkyl group or is an arylalkyl group having 1 to 12 C atoms; and
R⁴ is a hydrocarbon radical having 1 to 30 C atoms, which optionally contains ether oxygen atoms.

26. The aldazide of claim 24, wherein Z¹ is a radical of the formula (III), 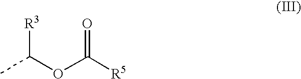
where R³ is a hydrogen atom or is an alkyl group or is a cycloalkyl group or is an arylalkyl group having 1 to 12 C atoms; and
R⁵ alternatively
is a hydrogen atom,
or
is a linear or branched alkyl radical having 1 to 30 C atoms, optionally with cyclic fractions and optionally with at least one heteroatom,
or
is a singly or multiply unsaturated, linear or branched hydrocarbon radical having 5 to 30 C atoms,
or
is an optionally substituted aromatic or heteroaromatic 5- or 6-membered ring.

27. The aldazide of claim 24, wherein Z¹ is a radical of the formula (IV), 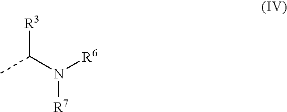
where
R³ is a hydrogen atom or is an alkyl group or is a cycloalkyl group or is an arylalkyl group having 1 to 12 C atoms; and
R⁶ and R⁷ either
independently of one another are each a monovalent aliphatic, cycloaliphatic or arylaliphatic radical having 1 to 20 C atoms and optionally containing heteroatoms in the form of ether oxygen or tertiary amine nitrogen,
or
together are a divalent aliphatic radical having 3 to 20 C atoms, which is part of an optionally substituted heterocyclic ring having 5 to 8 ring atoms and containing optionally, in addition to the nitrogen atom, further heteroatoms in the form of ether oxygen or tertiary amine nitrogen.

28. A method for reducing the yellowing of a cured polyurethane composition comprising using an aldazide of claim 24.

29. A method for reducing the outgassing of aldehyde or of ketone from a cured composition comprising providing hydrazide HY to a curable composition of claim 1.
